SEQUENCE LISTING (74 sequences)

SEQ ID NO: 1 - protein, 6 aa, Artificial Sequence
  Description of Artificial Sequence Synthetic peptide
LWLRRD

SEQ ID NO: 2 - protein, 7 aa, Artificial Sequence
  Description of Artificial Sequence Synthetic peptide
WTGDFDY

SEQ ID NO: 3 - protein, 268 aa, Artificial Sequence
  Description of Artificial Sequence Synthetic protein construct
EVQLQQSGAELVRPGTSVKLSCKALGYTFTDHEMHWVKQTPVHGLEWIGTIHPGSGVTAYNQKFKGKATLTADKSSSTAYMELSTLTSEDSAVYYCTPLWLRRDWGQGTTLTVSTGGGGSGGGGSGGGGSALDIQMTQTPKFMSTSVGDRVSVTCKASQNVATNVVWFQQKSGQSPKALIYSASYRYSGVPDRFTGSGSGTDFTLTISNVQSEDLAEYFCQQYNNYPLTFGAGTKLELKAAAGAPVPYPDPLEPRGAASAWSHPQFEK

SEQ ID NO: 4 - protein, 267 aa, Artificial Sequence
  Description of Artificial Sequence Synthetic protein construct
QLQQSGPELVKPGASVKISCKTSGYTFTEYTMHWVKQSHGKSLEWIGGIHPNNGDTSYNQRFKGKATLTVDKSSSTAYMELRSLTSEDSAVYYCARWTGDFDYWGQGTTLTVSTGGGGSGGGGSGGGGSALDIVMTQSPKFMSTSVGDRVSVTCKASQNVGTNVAWYQQKPGQSPKALIYSASYRYSGVPDRFTGSGSGTDFTLTISNVQSEDLAEYFCQQYNSYPYTFGGGTKLEIKAAAGAPVPYPDPLEPRGAASAWSHPQFEK

SEQ ID NO: 5 - DNA, 807 bp, Artificial Sequence
  Description of Artificial Sequence Synthetic nucleotide construct
gaggttcagctgcagcagtctggggctgagctggtgaggcctgggacttcagtgaagctgtcctgcaaggctttgggctacacatttactgaccatgaaatgcactgggtgaaacagacacctgtgcatggcctggaatggattggaactattcatccaggaagtggtgttactgcctacaatcagaagttcaagggcaaggccacactgactgcagacaaatcctccagcacagcctacatggagctcagcaccctgacatctgaagactctgctgtctattactgtacaccactttggttacgacgggactggggccaaggcaccactctcacagtgtcgacaggtggaggcggttcaggcggaggtggctctggcggtggcggaagtgcactcgatatccagatgacacagactccaaaattcatgtccacatcagtaggagacagggtcagcgtcacctgcaaggccagtcagaatgtggctactaatgtagtctggtttcaacagaaatcagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacaactatcctctcacgttcggtgctgggaccaagctggagctgaaagcggccgcaggtgcgccggtgccgtatccagatccgctggaaccgcgtggggccgcaagcgcttggagccacccgcagttcgaaaaataa

SEQ ID NO: 6 - DNA, 804 bp, Artificial Sequence
  Description of Artificial Sequence Synthetic nucleotide construct
cagctgcagcagtctggacctgagttggtgaagcctggggcttcagtgaagatatcctgcaagacttctggatacacgttcactgaatacaccatgcactgggtgaaacagagccatggaaagagccttgagtggattggaggtattcatcctaacaatggtgatactagctacaaccagaggttcaagggaaaggccacattgactgtagacaagtcatccagcacagcctacatggagctccgcagcctaacatctgaggattctgcagtctattactgtgcaagatggacgggggactttgactactggggccaaggcaccactctcacagtgtcgacaggtggaggcggttcaggcggaggtggctctggcggtggcggaagtgcactcgacattgtgatgacacagtctccaaaattcatgtccacatcagtaggagacagggtcagcgtcacctgcaaggccagtcaaaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatccgtacacgttcggaggggggaccaagctggaaataaaagcggccgcaggtgcgccggtgccgtatccagatccgctggaaccgcgtggggccgcaagcgcttggagccacccgcagttcgaaaaataa

SEQ ID NO: 7 - DNA, 3918 bp, Artificial Sequence
  Description of Artificial Sequence Synthetic nucleotide construct
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttggagccttttttttggagattttcaacgtgaaaaaattattattcgcaattcctttagttgttcctttctatgcggcccagccggccatggcccaggtccagtcgacaggtggaggcggttcaggcggaggtggctctggcggtggcggaagtgcactcatcaaacggcggccgcaggtgcgccggtgccgtatccggatccgctggaaccgcgtgccgcataggctggcggcggctctggtggtggttctggtggcggctctgagggtggcggctctgagggtggcggttctgagggtggcggctctgagggtggcggttccggtggcggctccggttccggtgattttgattatgaaaaaatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttctttgcctcagtcggttgaatgtcgcccttatgtctttggcgctggtaaaccatatgaattttctattgattgtgacaaaataaacttattccgtggtgtctttgcgtttcttttatatgttgccacctttatgtatgtattttcgacgtttgctaacatactgcgtaataaggagtcttaataagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcccggcgggtgtggtggttacgcgcagcgtgaccgctacacttgccagcgccctagcccccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgtcctttgacgttcgagtccacgttctttaatagtggactcttgttccaaactggaacaatactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 8 - DNA, 3351 bp, Artificial Sequence
  Description of Artificial Sequence Synthetic nucleotide construct
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgctcgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcatacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcaaattctatttcaaggagacagtcataatgaaatacctattgcctacggcagccgctggattgttattactcgcggcccagccggccatggcccaggtgcagctgcaggtcggcctcgagatcaaacgggcggccgcaggtgcgccggtgccgtatccagatccgctggaaccgcgtggggccgcaagcgcttggagccacccgcagttcgaaaaataataaggatccgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcccggcgggtgtggtggttacgcgcagcgtgaccgctacacttgccagcgccctagcccccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgtcctttgacgttcgagtccacgttctttaatagtggactcttgttccaaactggaacaatactcaaccctatctcgggctattcttttgatttataagggattttgccgatttcggcctattggttaaaaaatgagctgatttaacaaaaatttaacgcgaattttaacaaaatattaacgtttacaattttatggtgcagtctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcga

SEQ ID NO: 9 - DNA, 23 bp, Artificial Sequence
  Description of Artificial Sequence Synthetic primer
gaggtgcagcttcaggagtcagg

SEQ ID NO: 10 - DNA, 23 bp, Artificial Sequence
  Description of Artificial Sequence Synthetic primer
caggtgcagctgaaggagtcagg

SEQ ID NO: 11 - DNA, 23 bp, Artificial Sequence
  Description of Artificial Sequence Synthetic primer
gaggtccagctgcaacagtctgg

SEQ ID NO: 12 - DNA, 23 bp, Artificial Sequence
  Description of Artificial Sequence Synthetic primer
gaggttcagctgcagcagtctgg

SEQ ID NO: 13 - DNA, 23 bp, Artificial Sequence
  Description of Artificial Sequence Synthetic primer
caggtccaactgcagcagcctgg

SEQ ID NO: 14 - DNA, 23 bp, Artificial Sequence
  Description of Artificial Sequence Synthetic primer
caggttcagctgcagcagtctgg

SEQ ID NO: 15 - DNA, 23 bp, Artificial Sequence
  Description of Artificial Sequence Synthetic primer
gaggtgaagctggtggagtctgg

SEQ ID NO: 16 - DNA, 23 bp, Artificial Sequence
  Description of Artificial Sequence Synthetic primer
gaggtgaagctggtggaatctgg

SEQ ID NO: 17 - DNA, 23 bp, Artificial Sequence
  Description of Artificial Sequence Synthetic primer
gaggttcagcttcagcagtctgg

SEQ ID NO: 18 - DNA, 24 bp, Artificial Sequence
  Description of Artificial Sequence Synthetic primer
tgaggagacggtgaccgtggtccc

SEQ ID NO: 19 - DNA, 24 bp, Artificial Sequence
  Description of Artificial Sequence Synthetic primer
tgaggagactgtgagagtggtgcc

SEQ ID NO: 20 - DNA, 24 bp, Artificial Sequence
  Description of Artificial Sequence Synthetic primer
tgcagagacagtgaccagagtccc

SEQ ID NO: 21 - DNA, 24 bp, Artificial Sequence
  Description of Artificial Sequence Synthetic primer
tgaggagacggtgactgaggttcc

SEQ ID NO: 22 - DNA, 23 bp, Artificial Sequence
  Description of Artificial Sequence Synthetic primer
gacattgtgatgacacagtctcc

SEQ ID NO: 23 - DNA, 23 bp, Artificial Sequence
  Description of Artificial Sequence Synthetic primer
gatgttgtgatgacccaaactcc

SEQ ID NO: 24 - DNA, 23 bp, Artificial Sequence
  Description of Artificial Sequence Synthetic primer
gatatccagatgacacagactcc

SEQ ID NO: 25 - DNA, 23 bp, Artificial Sequence
  Description of Artificial Sequence Synthetic primer
caaattgttctcacccagtctcc

SEQ ID NO: 26 - DNA, 23 bp, Artificial Sequence
  Description of Artificial Sequence Synthetic primer
caggctgttgtgactcaggaatc

SEQ ID NO: 27 - DNA, 24 bp, Artificial Sequence
  Description of Artificial Sequence Synthetic primer
tttgatttccagcttggtgcctcc

SEQ ID NO: 28 - DNA, 24 bp, Artificial Sequence
  Description of Artificial Sequence Synthetic primer
ttttatttccagcttggtcccccc

SEQ ID NO: 29 - DNA, 24 bp, Artificial Sequence
  Description of Artificial Sequence Synthetic primer
tttcagctccagcttggtcccagc

SEQ ID NO: 30 - DNA, 24 bp, Artificial Sequence
  Description of Artificial Sequence Synthetic primer
acctaggacagtgaccttggttcc

SEQ ID NO: 31 - DNA, 56 bp, Artificial Sequence
  Description of Artificial Sequence Synthetic primer
gtcctcgcaactgcggcccagccggccatggccgaggtgcagcttcaggagtcagg

SEQ ID NO: 32 - DNA, 56 bp, Artificial Sequence
  Description of Artificial Sequence Synthetic primer
gtcctcgcaactgcggcccagccggccatggcccaggtgcagctgaaggagtcagg

SEQ ID NO: 33 - DNA, 56 bp, Artificial Sequence
  Description of Artificial Sequence Synthetic primer
gtcctcgcaactgcggcccagccggccatggccgaggtccagctgcaacagtctgg

SEQ ID NO: 34 - DNA, 56 bp, Artificial Sequence
  Description of Artificial Sequence Synthetic primer
gtcctcgcaactgcggcccagccggccatggccgaggttcagctgcagcagtctgg

SEQ ID NO: 35 - DNA, 56 bp, Artificial Sequence
  Description of Artificial Sequence Synthetic primer
gtcctcgcaactgcggcccagccggccatggcccaggtccaactgcagcagcctgg

SEQ ID NO: 36 - DNA, 56 bp, Artificial Sequence
  Description of Artificial Sequence Synthetic primer
gtcctcgcaactgcggcccagccggccatggcccaggttcagctgcagcagtctgg

SEQ ID NO: 37 - DNA, 56 bp, Artificial Sequence
  Description of Artificial Sequence Synthetic primer
gtcctcgcaactgcggcccagccggccatggccgaggtgaagctggtggagtctgg

SEQ ID NO: 38 - DNA, 56 bp, Artificial Sequence
  Description of Artificial Sequence Synthetic primer
gtcctcgcaactgcggcccagccggccatggccgaggtgaagctggtggaatctgg

SEQ ID NO: 39 - DNA, 56 bp, Artificial Sequence
  Description of Artificial Sequence Synthetic primer
gtcctcgcaactgcggcccagccggccatggccgaggttcagcttcagcagtctgg

SEQ ID NO: 40 - DNA, 36 bp, Artificial Sequence
  Description of Artificial Sequence Synthetic primer
gagtcattctcgtgtcgacacggtgaccgtggtccc

SEQ ID NO: 41 - DNA, 36 bp, Artificial Sequence
  Description of Artificial Sequence Synthetic primer
gagtcattctcgtgtcgacactgtgagagtggtgcc

SEQ ID NO: 42 - DNA, 36 bp, Artificial Sequence
  Description of Artificial Sequence Synthetic primer
gagtcattctcgtgtcgacacagtgaccagagtccc

SEQ ID NO: 43 - DNA, 36 bp, Artificial Sequence
  Description of Artificial Sequence Synthetic primer
gagtcattctcgtgtcgacacggtgactgaggttcc

SEQ ID NO: 44 - DNA, 41 bp, Artificial Sequence
  Description of Artificial Sequence Synthetic primer
tgagcacacagtgcactcgacattgtgatgacacagtctcc

SEQ ID NO: 45 - DNA, 41 bp, Artificial Sequence
  Description of Artificial Sequence Synthetic primer
tgagcacacagtgcactcgatgttgtgatgacccaaactcc

SEQ ID NO: 46 - DNA, 41 bp, Artificial Sequence
  Description of Artificial Sequence Synthetic primer
tgagcacacagtgcactcgatatccagatgacacagactcc

SEQ ID NO: 47 - DNA, 41 bp, Artificial Sequence
  Description of Artificial Sequence Synthetic primer
tgagcacacagtgcactccaaattgttctcacccagtctcc

SEQ ID NO: 48 - DNA, 41 bp, Artificial Sequence
  Description of Artificial Sequence Synthetic primer
tgagcacacagtgcactccaggctgttgtgactcaggaatc

SEQ ID NO: 49 - DNA, 48 bp, Artificial Sequence
  Description of Artificial Sequence Synthetic primer
gagtcattctcgacttgcggccgctttgatttccagcttggtgcctcc

SEQ ID NO: 50 - DNA, 48 bp, Artificial Sequence
  Description of Artificial Sequence Synthetic primer
gagtcattctcgacttgcggccgcttttatttccagcttggtcccccc

SEQ ID NO: 51 - DNA, 48 bp, Artificial Sequence
  Description of Artificial Sequence Synthetic primer
gagtcattctcgacttgcggccgctttcagctccagcttggtcccagc

SEQ ID NO: 52 - DNA, 48 bp, Artificial Sequence
  Description of Artificial Sequence Synthetic primer
gagtcattctcgacttgcggccgcacctaggacagtgaccttggttcc

SEQ ID NO: 53 - DNA, 17 bp, Artificial Sequence
  Description of Artificial Sequence Synthetic primer
ccccacgcggttccagc

SEQ ID NO: 54 - DNA, 17 bp, Artificial Sequence
  Description of Artificial Sequence Synthetic primer
tacctattgcctacggc

SEQ ID NO: 55 - DNA, 21 bp, Artificial Sequence
  Description of Artificial Sequence Synthetic oligonucleotide
ccagtcacttgtctggagctt

SEQ ID NO: 56 - DNA, 21 bp, Artificial Sequence
  Description of Artificial Sequence Synthetic oligonucleotide
gagcttgaagaagtgggtatt

SEQ ID NO: 57 - DNA, 21 bp, Artificial Sequence
  Description of Artificial Sequence Synthetic oligonucleotide
tgctggtggcattactggtgc

SEQ ID NO: 58 - DNA, 23 bp, Artificial Sequence
  Description of Artificial Sequence Synthetic oligonucleotide
gctgtcctggccaccccgtggaa

SEQ ID NO: 59 - DNA, 22 bp, Artificial Sequence
  Description of Artificial Sequence Synthetic oligonucleotide
ggcgcggagctgcggaatgcct

SEQ ID NO: 60 - DNA, 22 bp, Artificial Sequence
  Description of Artificial Sequence Synthetic oligonucleotide
cctcgctgaggatgttcgggtt

SEQ ID NO: 61 - DNA, 21 bp, Artificial Sequence
  Description of Artificial Sequence Synthetic oligonucleotide
cccgtgaacacagctgaggtt

SEQ ID NO: 62 - DNA, 23 bp, Artificial Sequence
  Description of Artificial Sequence Synthetic oligonucleotide
caaggtggacccacgagagcttt

SEQ ID NO: 63 - DNA, 30 bp, Artificial Sequence
  Description of Artificial Sequence Synthetic primer
cttacaaccacaatccctgggcacaatttt

SEQ ID NO: 64 - DNA, 24 bp, Artificial Sequence
  Description of Artificial Sequence Synthetic primer
ctttgtgggccctctgggctcaat

SEQ ID NO: 65 - DNA, 24 bp, Artificial Sequence
  Description of Artificial Sequence Synthetic primer
tgaaatgggcccgctgggctcaag

SEQ ID NO: 66 - DNA, 24 bp, Artificial Sequence
  Description of Artificial Sequence Synthetic primer
gggcttgggtattctaggctcgat

SEQ ID NO: 67 - DNA, 19 bp, Artificial Sequence
  Description of Artificial Sequence Synthetic primer
tcagaggaaggaaacaggt

SEQ ID NO: 68 - DNA, 20 bp, Artificial Sequence
  Description of Artificial Sequence Synthetic primer
ctggatggtgggaagatgga

SEQ ID NO: 69 - protein, 268 aa, Artificial Sequence
  Description of Artificial Sequence Synthetic protein construct
QVQLQQPGAVLVRPGASVKLSCKALGYTFTDHEMHWVRQTPVHGLEWIGAIHPGSGVTAYNQKFKGKATLTADKSSSTAYMELSILTSEDSAVYYCTPLWLRRDWGQGTTLTVSTGGGGSGGGGSGGGGSALDVVMTQTPKFMSTSVGDRVSVTCKASQNVGTNVAWYQQKPGQSPKALIYSASYRYSGVPDRFTGSGSGTDFTLTISNVQSEDLADYFCQQYNTYPLTFGAGTKLELKAAAGAPVPYPDPLEPRGAASAWSHPQFEK

SEQ ID NO: 70 - protein, 268 aa, Artificial Sequence
  Description of Artificial Sequence Synthetic protein construct
EVQLQQSGAELVRPGASVKLSCKALGYTFTDHEMHWVRQTPVHGLEWIGAIHPGSGVTAYNQKFKGKATLTADKSSSTAYMELSILTSEDSAVYYCTPLWLRRDWGQGTTLTVSTGGGGSGGGGSGGGGSALDIVMTQSPKFMSTSVGDRVSVTCKASQNVATNVVWYQQKSGQSPKALIYSASYRYSGVPDRFTGSGSGTDFTLTISNVQSEDLAEYFCQQYNSYPLTFGAGTKLELKAAAGAPVPYPDPLEPRGAASAWSHPQFEK

SEQ ID NO: 71 - DNA, 807 bp, Artificial Sequence
  Description of Artificial Sequence Synthetic nucleotide construct
caggtccaactgcagcagcctggggctgtactggtgaggcctggggcttcagtgaagctgtcctgcaaggctttgggctacacatttactgaccatgaaatgcactgggtgaggcagacacctgtacatggcctggaatggattggagctattcatccaggaagtggtgttactgcctacaatcagaagttcaagggcaaggccacactgactgcagacaaatcctccagcacagcctacatggagctcagtattctgacatctgaagactctgctgtctattactgtacaccactgtggttacgacgggactggggccaaggcaccaccctcacagtgtcgacaggtggaggcggttcaggcggaggtggctctggcggtggcggaagtgcactcgatgttgtgatgacccaaactccaaaattcatgtccacatcagtaggagacagggtcagcgtcacctgcaaggccagtcagaatgtgggtactaatgtagcctggtatcaacagaaaccagggcaatctcctaaagcactgatttactcggcatcctaccggtacagtggagtccctgatcgcttcacaggcagtggatctgggacagatttcactctcaccatcagcaatgtgcagtctgaagacttggcagactatttctgtcagcaatataacacctatcctctcacgttcggtgctgggaccaagctggagctgaaagcggccgcaggtgcgccggtgccgtatccagatccgctggaaccgcgtggggccgcaagcgcttggagccacccgcagttcgaaaaataa

SEQ ID NO: 72 - DNA, 807 bp, Artificial Sequence
  Description of Artificial Sequence Synthetic nucleotide construct
gaggttcagctgcagcagtctggggctgagctggtgaggcctggggcttcagtgaagctgtcctgcaaggctttgggctacacatttactgaccatgaaatgcactgggtgaggcagacacctgtacatggcctggaatggattggagctattcatccaggaagtggtgttactgcctacaatcagaagttcaagggcaaggccacactgactgcagacaaatcctccagcacagcctacatggagctcagtattctgacatctgaagactctgctgtctattactgtacaccactgtggttacgacgggactggggccaaggcaccactctcacagtgtcgacaggtggaggcggttcaggcggaggtggctctggcggtggcggaagtgcactcgacattgtgatgacacagtctccaaaattcatgtccacatcagtaggagacagggtcagcgtcacctgcaaggccagtcagaatgtggctactaatgtagtctggtatcaacagaaatcagggcaatctcctaaagcactgatttactcggcatcctaccggtacagtggagtccctgatcgcttcacaggcagtggatctgggacagatttcactctcaccatcagcaatgtgcagtctgaagacttggcagagtatttctgtcagcaatataacagctatcctctcacgttcggtgctgggaccaagctggagctgaaagcggccgcaggtgcgccggtgccgtatccagatccgctggaaccgcgtggggccgcaagcgcttggagccacccgcagttcgaaaaataa

SEQ ID NO: 73 - DNA, 21 bp, Artificial Sequence
  Description of Combined DNA/RNA Molecule Synthetic oligonucleotide
caacuuuaaucugcaacgutt

SEQ ID NO: 74 - DNA, 21 bp, Artificial Sequence
  Description of Combined DNA/RNA Molecule Synthetic oligonucleotide
ttguugaaauuagacguugca

(19) United States
(12) Patent Application Publication
(10) Pub. No.: US 2009/0215175 A1
(43) Pub. Date: Aug. 27, 2009

(54) MODULATION OF THE POLIOVIRUS RECEPTOR FUNCTION

(75) Inventors: Christine Margarete UNGER, Munchen (DE); Gerald BESTE, Great Shelford (GB); Carolin ZEHETMEIER, Munchen (DE); Blanca LAIN, Allston, MA (US); Claudia TORELLA, Munchen (DE); Daniel G. JAY, Brighton, MA (US); Brenda K. EUSTACE, Brookline, MA (US); Kevin Ernest SLOAN, Jamaica Plain, MA (US)

Correspondence Address: DAVID S. RESNICK, NIXON PEABODY LLP, 100 SUMMER STREET, BOSTON, MA, 02110-2131, US

(73) Assignee: TRUSTEES OF TUFTS COLLEGE, MEDFORD, MA (US)

(21) Appl. No.: 12/269,137

(22) Filed: Nov. 12, 2008

Related U.S. Application Data
(63) Continuation of application No. 10/546,703, filed on Feb. 15, 2006, now abandoned.
(60) Provisional application No. 60/450,064, filed on Feb. 24, 2003.

(30) Foreign Application Priority Data
May 28, 2003 (EP) 03012314.5

(51) Int. Cl. C12N 5/00

(52) U.S. Cl. 435/377

(57) ABSTRACT

The present invention relates to the identification, the isolation and the use of molecules interfering with the function (s) mediated by the poliovirus receptor (PVR) on cells. The molecules can be used for the treatment of cells having a metastatic potential, metastasis and cancer. Further methods are provided that are useful for identifying and isolating molecules, which have the capacity to modulate PVR mediated adhesion or invasion potential of cells.

### CROSS REFERENCE TO RELATED APPLICATIONS

The present application is a continuation of U.S. Ser. No. 10/546,703 filed on Feb. 15, 2006, which is a 371 U.S. National Entry of International Application No. PCT/EP2004/001637 filed on Feb. 19, 2004, and which claims the benefit under 35 U.S.C. § 119(e) of U.S. Provisional Application Ser. No. 60/450,064 filed on Feb. 24, 2003 and which claims the benefit under 35 U.S.C. § 119(b) of European Application No. 03012314.5 filed on May 28, 2003, the contents of which are herein incorporated by reference in its entirety.

### GOVERNMENT SUPPORT

This invention was made with government support under CA81668 awarded by the National Institute of Health. The government has certain rights in the invention.

### BACKGROUND OF THE INVENTION

Malignant tumors shed cells, which migrate to new tissues and create secondary tumors. The process of generating secondary tumors is called metastasis and is a complex process in which tumor cells colonize sites distant from the primary tumor. Liotta ((1986) Cancer Res. 46, 1-7) has proposed a three-step hypothesis for the process of metastasis: The first step is tumor cell attachment via cell surface receptors. The anchored tumor cell next secretes hydrolytic enzymes or induces host cells to secrete enzymes, which can degrade the matrix locally. Matrix lysis most likely takes place in a highly localized region close to the tumor cell surface. The third step is tumor cell locomotion into the region of the matrix modified by proteolysis. Recently, research has been focused on identifying specific proteins involved in metastasis, which can be used as a basis for better diagnostic or improved therapeutic strategies. Cell adhesion molecules (CAM's), which mediate cell-cell or cell-matrix interactions, have been proposed to be involved in the process of metastasis. Cell adhesion in normal cells involves interactions between numerous cell surface proteins. Specifically, adhesive interactions are known to involve interactions between substances surrounding the cell and extracellular adhesion receptors. It has also become apparent that cell adhesion molecules fulfill much more complex functions which may result in cells acquiring the ability to proliferate and invade host tissues. Cell adhesion receptors comprise molecules such as selecting, cadherins, integrins and immunoglobulins.

The integrin receptor family has been studied intensively and it is well-known that the adhesion receptor alpha v beta 3 integrin contributes to tumor cell functions and is potentially involved in melanoma growth and metastasis. Felding-Habermann et al (Clin Exp Metastasis (2002) 19, 427-36) showed that expression of integrin alpha v beta 3 promotes the metastatic phenotype in human melanoma by supporting specific adhesive, invasive and migratory properties of the tumor cells. Inhibition of alpha v beta 3 expressed on melanoma cells reduced metastasis significantly and further prolonged animal survival. Haier et al showed that the ability of tumor cells to adhere to extracellular matrix proteins is an important factor in metastasis formation (Haier et al (2002) J. Exp. Ther. Oncol. 2, 237-45 and Haier et al. (1999) Br. J. of Cancer 80, 1867-74).

The process of metastasis formation also depends on the invasiveness of tumor cells. Invasiveness describes the potential of a tumor cell to migrate and disregard the extracellular matrix. The extracellular matrix consists of a supramolecular aggregate of connective tissue proteins including collagens, elastin, fibronectin, laminin, glycoproteins and glycosaminoglycans. It has normally supportive and nutritive function for development and organization of tissue, but also serves as a physical barrier to limit the migration of normal cells. Although considerable information is available on the digestion of isolated connective tissue proteins by tumor enzymes, less is known about the mechanisms by which tumor cells penetrate highly complex mixtures of extracellular matrix proteins.

### OBJECT OF THE INVENTION

It was therefore one object of the invention to identify and isolate agents modulating the adhesion and/or invasion behavior of cancer or metastatic cells preferably via a receptor, which is predominantly expressed on tumor cells.

It was the achievement of the inventors of the present invention to establish an experimental set up including various functional assays to identify and eventually isolate molecules, which have as main characteristic the capability to modulate the adhesion and/or invasion behavior of a cell. In additional experiments the inventor could specify the involved receptor molecule, a poliovirus receptor (PVR), derivative and/or analog thereof, which was involved in the adhesion and invasive behavior of a cell and which was modulated by the molecules according to the present invention.

The PVR is a transmembrane glycoprotein with a N-terminal signal sequence, three extracellular immunoglobulin (Ig)-like domains, a transmembran domain and a cytoplasmic tail. It can be also described as a member of the immunoglobulin superfamily having a molecular size of approximately 80 kDa and a specific structure composed of three Ig-like domains, specifically an outermost V-like domain followed by two C2-like domains. An alternative nomenclature for PVR is CD155.

Originally, PVR was discovered as the cellular receptor for poliovirus, the causative agent for poliomyelitis. The interaction between the poliovirus and PVR was studied in great detail (see e.g. Koike et al. (1991) Proc. Nat. Acad. Sci. USA 88, 4104-08; Belnap et al. (2000) PNAS 97, 73-78; Racaniello (1996) Structure 4, 769-73; Racaniello (1996) Proc. Natl. Acad. Sci. USA 93, 11378-81).

Nonetheless, the physiological function (s) of PVR remained largely unknown and the biological importance of PVR just slowly begins to be elucidated. Solecki et al. ((2002) J. Biol. Chem. 277, 25697-700) recently discussed PVR/CD155 as being involved in mediating cell adhesion to extracellular matrix molecules as well as being up-regulated in neuroectodermal tumors. An analysis of the transcriptional regulation of CD155 revealed two potent regulators (activator protein-2 and nuclear respiratory factor-1) of the CD155 transcription. Both transcription factors are expressed during CNS development and may direct the developmental CD155 expression. The over-expression of CD155 in neuroectodermal tumors suggests the existence of gene regulatory pathways in neuroectodermal tumors, which reactivate CD155 expression.

Mason et al. ((2001) Gut 49, 236-40) revealed that the CD155 gene is also overexpressed in colorectal tumors and showed that the overexpression begins at very early stage in tumorigenesis and continues to late stages.

Lange et al. ((2001) Virol. 285, 218-227) further report a mediation of cell adhesion by the various polio receptor related (PRR) proteins and a specific CD155/vitronectin interaction, which seems to be required for the establishment of a proper immune response inside the germinal centers of secondary lymphoid tissue.

Nonetheless, the physiological function of CD155/PVR is not fully understood. The determination of the physiological role of a protein is a prerequisite for deciding whether interference with this protein's function might be a possible avenue for the treatment of disease or not. It must be kept in mind that in a physiological setting, which means e.g. in a naturally occurring tumor cell of a patient, PVR is overexpressed. Such overexpression may allow a cell to react differently to stimulation, and thereby can modulate and change physiological PVR function.

Accordingly, it was a further object of the present invention to provide means and methods to modulate the functions of PVR and thereby to contribute to a better understanding of the role of PVR.

Additionally, it was a further object of the present invention to develop pharmaceutical compositions or drugs, which inhibit PVR mediated functions such as adhesiveness and/or invasiveness of cells.

### SUMMARY OF THE INVENTION

The present invention provides for the first time molecules, which modulate the PVR functions necessary for adhesion, trafficking, invasion and/or a metastatic potential of cells. These molecules are selected from the group comprising small chemical compounds, oligonucleotides, peptides, oligopeptides, polypeptides, proteins, antibodies, antibody fragments, anti-idiotypic antibodies and/or bioconjugates.

One feature of these molecules is that each of them binds specifically to one or more intra- or extracellular domains of the PVR, CD155 or any derivative or analog thereof, which is expressed on cells being involved in a proliferative disease or disorder, having a metastatic potential or deriving from a naturally occurring tumor.

This specific binding itself and/or the activation or induction of a label, which is attached to the molecule, starts the modulation of the PVR function (s). In other words due to a blocking and/or destruction of an active site in the PVR the mediated function (s), which influence the adhesion, trafficking or invasion behavior of a cell, are either induced, increased, stabilized, strengthened, prevented, inhibited, decreased and/or diminished.

In an unbiased screen for molecules that can inhibit adhesiveness and invasiveness of naturally occurring tumor cells surprisingly specific antibody fragments binding to the extracellular domain of PVR and having the amino acid sequence SEQ ID No.: 3 or SEQ ID No.: 4 has been identified as such a modulator.

The present invention further comprises peptides, polypeptides, proteins, antibodies or antibody fragments comprising the amino acid sequence LWLRRD (SEQ ID No.: 1) and/or WTGDFDY (SEQ ID No.: 2), which can specifically bind to the extracellular domain of PVR and inhibit the adhesion and/or invasive behavior of a cell.

The present invention relates further to the DNA sequences (SEQ ID No.: 5 or 6) translating to SEQ ID No.: 3 or SEQ ID No.: 4, to DNA sequences corresponding or homologous to SEQ ID No.: 5 or 6, but also to the DNA sequences, which have a degenerated code but still translate into the amino acid sequences as above.

In a further embodiment the invention relates to nucleic acid molecules encoding any of the molecules or polypeptides of the invention.

The invention further provides expression vectors encoding any of the DNA sequences as above, as well as to host cells comprising such a vector. Still further the invention provides a pharmaceutical composition, a bioconjugate or a kit comprising any of the amino acid sequences, DNA sequences or vectors as above.

The invention provides the molecules as above to modulate or inhibit PVR function as medicament or drug and/or for the manufacturing of a medicament or drug for the treatment or prevention of adhesion, trafficking, invasion and/or metastatic potential of naturally occurring cancer cells, wherein adhesiveness, invasiveness and/or metastatic potential of said cancer cells depend on PVR mediated function.

In a further embodiment the invention relates to a method for the identification of a ligand useful for inhibiting the adhesiveness or invasiveness of a naturally occurring cancer cell, particularly the identification of such ligands that bind to the extracellular domain of PVR. This method comprises as a first step the establishment of a ligand library. Typical ligands represented in such a library are small chemical compounds, oligonucleotides, peptides, oligopeptides, polypeptides, proteins, antibodies, antibody fragments, anti-idiotypic antibodies and/or bioconjugates. The library is subsequently screened for tumor-specific ligands, which are further tested to identify ligands, which modulate the PVR function (s) in assays further provided by the present invention.

In a further embodiment the invention provides several diagnostic methods to determine adhesiveness and/or invasiveness and, thus, the metastatic potential of a naturally occurring cancer cell comprising the isolation of naturally occurring cancer cells, applying the assay to determine the adhesiveness or invasiveness of the cells and analyzing the results compared to normal cells.

In a further embodiment the invention relates to a method of treating or preventing adhesion, trafficking, invasion and/or metastatic potential of cells in a patient, wherein the method comprises administering to a person in need thereof a therapeutically effective amount of the molecules, the vectors and/or the pharmaceutical composition according to the invention.

### DETAILED DESCRIPTION OF THE INVENTION

In order that the invention described herein may be more fully understood, the following detailed description and definitions are provided.

A solution to the object of the present invention, namely to modulate the adhesion, trafficking and/or invasion behavior of a cell is achieved by the features of the independent claims.

The molecules, which had been identified by the inventors, have a common characteristic as they all bind specifically to the PVR receptor or any of its derivatives. The molecules or ligands specifically binding to the PVR according to the invention bind the PVR or any of its derivatives regardless of the physiological set up, thus on a naturally presented receptor, an isolated receptor as well as on a column-bound receptor.

The term “PVR” as used herein comprises the originally discovered Poliovirus receptor, which correlates with the CD155 molecule. PVR as used herein further comprises any member of the known family of related PVR's, which originate either by alternative RNA splicing or as transcripts of related homologous genes, namely human PRR1, PRR2 and PRR3 gene (Racaniello (1996) Proc. Natl. Acad. Sci. USA, Vol. 93 pp 11378-81; Lange et al. (2001) Virol. 285, 218-227). The abbreviation PVR is used hereinafter even if the whole family of related receptors, like CD155, PRR1 to PRR3, or derivatives, analogs or fragments thereof are addressed.

The molecule according to the invention binds to one or more intra- or extracellular regions or domains of the PVR. The extracellular region of PVR is defined as that part of the PVR protein outside of the cellular membrane, particularly the extracellular amino acid loops between amino acid 26 and amino acid 854. More specifically the V-like domain and the two C2-like domains. Thus, molecules according to the invention specifically recognize one or more epitopes of PVR, or epitopes of conserved variants of PVR as listed above.

The term “epitope” as used herein includes any protein determinant capable of specific binding to an immunoglobulin or an antibody fragment. Epitopic determinants usually consist of chemically active surface groupings of molecules such as exposed amino acids, amino sugars, or other carbohydrate side chains and usually have specific three-dimensional structural characteristics, as well as specific charge characteristics. It should be appreciated that PVR is a glycoprotein, so not only the amino acid sequence, but also the sugar modifications on it are considered as being epitopes or recognisable part of the extracellular region of PVR.

The molecules according to the present invention are characterized by an additional functional definition, namely, that they have all the ability to specifically bind the PVR as a ligand e.g. on a living or fixed cell. A ligand “binding specifically to a PVR” as mentioned herein can be a ligand, which binds to PVR under the buffer conditions given in the Examples. The dissociation constant between the ligand and PVR can be measured, e.g. by use of the so-called BIACORE System (see, for example, Fivash et al. Curr Opin Biotechnol. (1998) 9, 97-101) and “binding specifically” can then be understood to mean that the dissociation constant between the ligand and PVR is lower than 10 LM, preferably lower than 1, uM, more preferably lower than 500, 400, 300, 200, 100, 50, 20 nM, most preferable from 0.1 nM to 20 nM if measured under standard conditions, for example at 20° C., ambient pressure and in a suitable buffer, e.g. 20 mM Tris, 100 mM NaCl, 0.1 mM EDTA at pH of 7.0.

Further, the molecules according to the present invention have the ability to modulate the physiological functions and/or interactions of PVR. A “molecule modulating PVR function”, is a molecule resulting in modulation of the biological activity of PVR, which are for example a mediation of adhesiveness to extracellular matrix structures of the cell expressing PVR, a mediation of trafficking through normal adjacent or distant tissue, organs and/or extracellular matrix structures of the cell expressing PVR, and/or a mediation of invasiveness of the cell expressing PVR into normal adjacent or distant tissue, organs or extracellular matrix structures. This modulation of the biological activity of PVR can be assessed by measuring one or more indicators of PVR's biological activity, such as PVR dependent invasiveness or PVR dependent adhesiveness. These indicators of PVR's biological activity can be assessed by in in vitro or in vivo assays (see Examples). The ability of a molecule to inhibit PVR activity is primarily assessed by inhibition of PVR-induced adhesiveness compared with adhesion of invasive human cancer cells (e.g. sarcoma cells, breast cancer cells).

A molecule modulating PVR function of the invention is not a molecule which is a general inhibitor of protein function, like a protease, like a denaturing agent, e.g. urea or guanidinium hydrochloride, like heavy metal atoms or like small molecules (e.g. aldehydes or isocyanates) reacting covalently and non-specifically with biomolecules (lipids, proteins, sugars). A molecule modulating PVR function is characterized—inter alia—by its ability to modulate PVR function at a concentration at which it does not inhibit the function of the insulin receptor (e.g. as determined in an anti-<BR><BR> Phosphotyrosine Western Blot Assay, see e.g. B. Cariou et al. (2002) J Biol Chem., 277, 4845-52) or that of the acetylcholin receptor (e.g. as determined by measuring the Ca influx, see M. Montiel et al. (2001) Biochem Pharmacol. 63, 337-42.) or that of the B-CAM cell surface glycoprotein (e.g. by determining binding of hemoglobin A red blood cells (AA RBCs) to immobilized laminin as described in the section “Flow chamber assays” on page 2551 of Udani et al. (1998) J. Clin. Invest. 101, 2550-2558). Only a molecule modulating PVR function but at the same concentration not significantly affecting the function of the other three receptors mentioned is a molecule as used in this patent.

According to one embodiment the molecule modulating the PVR function binds to the PVR, preferably—but not exclusively—to an extracellular part of the PVR, in a way that the whole receptor or at least one or more active domains of the receptor are covered by the molecule and, thus, are not available for other ligands. Accordingly, since the receptor or its active domains are covered no receptor-mediated functions can occur.

According to another embodiment the molecule modulating the PVR function binds to the PVR, preferably—but not exclusively—to an extracellular part of the PVR, in a way that the only one or more active domains of the receptor are bound by the molecule and, thus, blocked from interacting with other ligands. Accordingly, no naturally occurring receptor-mediated functions can be induced.

According to another embodiment the molecule modulating the PVR function binds to the PVR, preferably—but not exclusively—to an extracellular part of the PVR, in a way that the molecule binds close to an active domain and, thus, interferes with and/or disturbs the binding of other ligands. Accordingly, no or only modulated receptor-mediated functions can occur.

According to another embodiment the molecule modulating the PVR function binds to the PVR, preferably—but not exclusively—to an extracellular part of the PVR, in a way that the molecule by binding rearranges extracellular domains of the receptor and, thus, interferes with and/or disturbs the binding of other ligands. Accordingly, no or only modulated receptor-mediated functions can occur.

According to another embodiment the molecule modulating the PVR function binds to the PVR, preferably—but not exclusively—to an extracellular part of the PVR, in a way that the molecule by binding destroys one or more active domains or even the whole receptor and, thus, interferes with and/or disturbs the binding of other ligands.

Accordingly, no or only modulated receptor-mediated functions can be induced.

According to another embodiment the molecule modulating the PVR function binds to the PVR, preferably—but not exclusively—to an extracellular part of the PVR, in a way that the molecule by binding activates and increases the receptor mediated function (s).

According to another embodiment the molecule modulating the PVR function binds to the PVR, preferably—but not exclusively—to an extracellular part of the PVR, in a way that the molecule by binding promotes further binding of molecules to the receptor or one or more active domains thereof. In this case the receptor-mediated functions are increased, accelerated and/or prolonged.

The present invention further provides a method of identifying a ligand, binding specifically to the extracellular region of PVR, by screening a naive or immune library, preferably a phage display library, wherein said ligand is capable of affecting a biological function, like adhesiveness and invasiveness, of PVR, comprising the steps of
a) contacting a phage library of ligands with cancer cells;
b) separating said cancer cells and the ligands bound thereto from ligands not bound to said cells;
c) removing phages bound unspecifically to said cells, e.g. by washing said cells with a buffered detergent solution, under conditions where said cells do not lyse;
d) eluting phages bound to said cells; and
e) determining the identity of the ligand represented by said eluted phages
f) testing the ligand in a biochemical or biological assay for its capability to interfere with PVR function.

The identity of phages representing the ligand obtained with step e) can be determined by, e.g., sequencing the DNA encoding the ligand in case the ligand is an antibody or antibody fragment, or, in the case of a commercial ligand library with gridded or numbered phages, by determining the grid position or the number of the phage. The grid position or the number then can reveal the identity of the ligand represented by the phage.

After step d) the pool of phages is enriched in phages binding to PVR. Those phages binding to PVR can finally be identified by numerous methods known in the art. Phages can be separated to form individual clones and the clones of the phages can be probed with labeled PVR protein, or a labeled part of the PVR protein, e.g. an at least seven amino acid long peptide of the extracellular region of PVR. Clones binding to such a probe are identified as PVR-binders. Phages can also be affinity purified on purified PVR protein or on recombinant PVR.

Alternatively, in case the ligand is e.g. an antibody or antibody fragment, the open reading frame encoding the antibody or antibody fragment can be recloned from the whole enriched pool into an expression vector, the antibody or antibody fragment can then be expressed in clones of another host cell, and the clone of the host cell carrying the expression vector comprising a nucleic acid encoding for the antibody or antibody fragment binding specifically to PVR can be identified, e.g. by the method described above for the identification of relevant phage clones, by the method of Examples 2 and 3, or by affinity purification on recombinant PVR.

A particular advantage of this method is that the ligand specific for the accessible part of the extracellular region of PVR is obtained, since the initial selection step is performed on intact cells, which present the accessible part of the extracellular region of PVR for binding of the phages.

In another preferred embodiment of the invention the method comprises an additional step of screening on recombinant PVR protein.

The method comprises instead of step e) the further steps of:
e) contacting isolated phages with recombinant PVR;
f) washing said PVR with a buffered detergent and/or high salt solution; and
g) eluting phages bound to PVR; and
h) determining the identity of the ligand represented by said eluted phages.

In certain embodiments, the ligand expressed on the phages comprise an antibody or an antibody fragment selected from the group consisting of scFv, dsFv, Fab′, Fab, F (ab′)2, Fv, single domain antibodies (DABs) and diabodies, more particularly selected from the group consisting of scFv, dsFv, Fv, single domain antibody or diabody, still more particularly selected from the group consisting of scFv, single domain antibody or diabody and even more preferably a scFv.

Phages useful for phage display can be all phages that can be obtained from culture and that are amenable to genetic engineering techniques and are capable of displaying a foreign ligand on their surface. Phage display has been disclosed in Smith et al. (1985) Science, 228, 1315-17, phage display of an antibody has been disclosed in WO 91/17271.

The “detergent” used in steps c) and f) is a detergent solution, preferably buffered, and can be Tween in a concentration of 0.001-0.5%, particularly 0.01-0.1%. “High salt” in step f) is a high salt solution, preferably buffered, and has an ionic strength of 10 mM-1M, particularly 20-500 mM, more particularly 50-350 mM, even more preferably 80-250 mM. Typical useful anions are, for example, chloride, citrate, phosphate, hydrogen phosphate or borate. Typical useful cations are, for example, sodium, potassium, lithium, calcium or magnesium.

The buffered solution in the above paragraph typically has a pH of 7-8. For example, DMEM or PBS, particularly with 1-20%, more particularly 5-15%, even more preferably about 10% FCS, can be used as buffers.

Isolation of cells with phages bound to them is effected by gentle centrifugation at g values from 200 to 300 for 3 to 20 minutes, particularly 5 to 10 minutes. Elution of bound phages, both to cells and to immobilized PVR, is effected by a wash with 2-100 mM, particularly 4-50 mM, more particularly 5-20 mM, even more preferably around 10 mM glycine at a pH of from 0 to 2.5, particularly from 1 to 2.5, more particularly from 1.5 to 2.5.

The PVR interfering or inhibiting functionality of these ligands, particularly of these antibodies or antibody fragments may be assayed as described above, in vivo or in a cell culture experiment. Cell culture assays include assays that determine the inhibitory effect of the ligands of this invention in an invasion, migration or adhesion assay as described in Examples 8 and 9, 9.1-9.3 or Examples 11 and 12. Results of the different assays are provided in the Figures.

The method advantageously combines a screening step based on binding to the surface of a cell with a screening step based on a functional assay, which identifies ligands having the capability to interfere or inhibit PVR function.

In a preferred embodiment the ligand is capable of inhibiting a biological function of PVR. For example, the biological function of a PVR can be invasion, adhesion, proliferation or angiogenesis. An invasion or migration assay measures the invasiveness of a cell. An invasion assay is, e.g., the assay performed in Example 8 and 9, 9.1 and 9.2, a migration assay is described in Example 9.3 of this application. An adhesion assay is described in Examples 11 and 12 of this application.

The term “invasiveness” as used herein is the ability of a cell to migrate through a layer of other cells or to migrate through the extracellular matrix. Invasiveness can be assessed by the Matrigel assay as described in the Examples. According to the present invention invasion is measured as cells that reach the lower surface of the filter during a certain incubation period. When more than 40% of cells within 6 to 12 hours reach the other side of the filter and form colonies in an invasion assay like the one described in the Examples, the naturally occurring cancer cell is defined as invasive. The control cells forming only 5% colonies in the same time frame are defined as non-invasive.

The term “adhesiveness” as used herein is the ability of a cell to reattach after they have been removed from the matrix on which it had been grown, re-suspended as a solution of single cells (not in direct contact with other cells of the solution), and replated on a matrix to which adhesion is possible. A cell is defined as adhesive if in an assay as described in the Examples, more than 40% of the cells adhere within a time of 30 to 120 minutes. In contrast, only 5% of the control cells adhere within the same time frame.

The term “angiogenesis” is the process where cells induce formation of new blood vessels in their proximity. Angiogenesis can accompany the growth of malignant tissue and can therefore be a property of malignant cells. That is to say that malignant cells can have the property of inducing angiogenesis.

Preferably, the biological function—such as adhesiveness and invasiveness of a cancer cell—to be inhibited by a ligand, e.g. an antibody or antibody fragment, is identified by the method of the present invention.

The term “inhibition” is understood to be at least a 20%, preferably a 30%, more preferably a 50%, even more preferable a 60% decrease in function, as defined by PVR function in an invasion assay as mentioned above, when compared to a negative control with the same experimental conditions, but without the molecule of the invention. A molecule is defined as not significantly affecting the function of the other three receptors if the decrease in function affected by the molecule of the invention is less than 20%, more preferably less than 15%, even more preferably less than 10%.

Additionally, the polypeptide, the antibody or antibody fragment according to the present invention is considered to inhibit the biological function of PVR if a reduction of the adhesiveness and of the invasiveness of naturally occurring cancer cells in an experiment by more than 20%, preferably more than 60%, can be detected.

Additionally, in the case of a molecule of the invention which inhibits gene expression of PVR, such a molecule decreases PVR expression by more than 50%, preferably by more than 80%, still more preferably by more than 90%, most preferably by more than 95% when measured in a quantitative western blot normalized to the level of beta tubulin, when present in an experiment at a concentration of 10 nM to 100 u. M, preferably at around 1 u. M, in which the amount of PVR is compared between two otherwise identical samples, wherein in one sample the molecule of the invention was allowed to inhibit PVR expression. In the same experiment the molecule of the invention does not decrease the amount of the beta tubulin present per cell by more than 20%, and said molecule does not decrease the relative level of the insulin receptor and the B-CAM cell surface glycoprotein by more than 20%.

With the method, the inventors provided for the first time a possibility to identify and subsequently isolate molecules, which modulate PVR function (s). Furthermore, by attaching selected labels, which can be induced on command, e.g. a laser or a light beam, the inventors were able to modify the selected molecules in a way that the selected molecules were able to inhibit the PVR mediated adhesion behavior of a cell by about 41% to 55% (see, FIG. 3). Furthermore, the PVR mediated invasion behavior of cells was inhibited by at least 25% compared with untreated cells (FIGS. 2a, 2b, 2c or 2d).

According to an embodiment of the present invention molecules identified and/or isolated due to their specific binding capacity to PVR and due to their ability to modulate PVR functions are selected from groups comprising small chemical compounds, oligonucleotides, peptides, oligopeptides, polypeptides, proteins, antibodies, antibody fragments, anti-idiotypic antibodies and/or bioconjugates.

A “small chemical compound” as used in this invention is a molecule with a molecular weight between 50 Da and 10000 Da, preferably between 100 Da and 4000 Da, more preferably between 150 Da and 2000 Da, or a physiologically acceptable salt thereof.

Additionally, in the case of a small chemical compound of the invention, said compound is considered to modulate or inhibit the biological function of PVR if it reduces the adhesiveness of naturally invasive cancer cells by more than 30%, preferably by more than 60% and thereby not affecting cell morphology, cell cycle progression (determined by analyzing the DNA content of a cell population by propidium iodide staining and FACS analysis), and not increasing the percentage of the cells of the culture that show signs of apoptosis (determined by measuring the percentage of cells showing DNA fragmentation, e.g. by a so called tunnel-assay).

A “polypeptide” as used herein is a molecule comprising more than 10, preferably more than 20, most preferably more than 30, and less than 10000, more preferably less than 2500, most preferably less than 1000 amino acids. Also polypeptides, which contain a low percentage of modified or non-natural amino acids, are encompassed.

For amino acid sequence above 10000 residues the term “protein” is used hereinafter, for amino acid sequence with less than 10 residues the term “peptide” is used. Also peptides or proteins, which contain a low percentage of modified or non-natural amino acids, are encompassed.

The terms “antibody” and “immunoglobulin”, as used herein refer to any immunological binding agent, including polyclonal and monoclonal antibodies. Normal antibodies have a common core structure of two identical light chains and two identical heavy chains. One light chain is attached to each heavy chain and the two heavy chains are attached to each other. Both the light and the heavy chains contain series of repeating, homologous units each about 110 amino acid residues in length, which fold independently in the so-called immunoglobulin (Ig) domain. To provide an unlimited and high specificity of antibodies against antigens and the correlating diversity of structure the N-terminal end of the light and heavy chains encompasses a so-called variable (V) region to distinguish from the more conserved constant (C) region of the remainder of each chain. Each V comprises three hypervariable regions, which are called also “complementarity-determining regions” (CDR) since these sequences form a surface complementary to the three-dimensional surface of the bound antigen. The CDRs of an antibody are the parts of these molecules that determine their specificity and make contact with specific ligands. The CDRs are the most variable parts of the molecule and contribute to the diversity of these molecules. They are structurally defined in a human IgG as amino acids 24 to 41 (CDR-L1), 50 to 57 (CDR-L2) and 90 to 101 (CDR-L3) of the light chain and amino acids 26 to 38 (CDR-H1), 51 to 70 (CDR-L2) and 100 to 125 (CDR-H3) of the heavy chain (see Kabat et al. (1987) 4th edn US Department of Health and Human Services, Public Health Service, NIH, Bethesda). The CDR regions of an antibody fragment can easily be determined by somebody skilled in the art by aligning the antibody fragment with said human IgG, e.g. using a program of the NCBI that allows to “Blast”, and thereby align, two sequences with one another, and identifying the amino acids of the antibody fragment corresponding to the CDRs of a human IgG.

Antibodies according to the present invention may be also selected from modified immunoglobulins, for example chemically or recombinantly produced antibodies, CDR grafted antibodies or humanized antibodies, site-directed-mutagenized antibodies.

In a further embodiment the molecule of the invention is an antibody fragment, in particular a single chain antibody fragment (scFv), dsFv, Fab′, Fab, F (ab′)2, Fv, single domain antibody and/or diabody.

The term “antibody fragment” is used to refer to any fragment of an antibody-like molecule that has an antigen binding region, and this term includes antibody fragments such as scFv, dsFv, Fab′, Fab, F (ab′)2, Fv, single domain antibodies (DABs), diabodies, and the like. The techniques for preparing and using various antibody-based constructs and fragments are well known in the art (see Kabat et al. (1991) J. Immunol. 147, 1709-19).

“Single chain antibody fragments” (scFv) comprise the variable domain of the heavy chain (VH) and the variable domain of the light chain (VL) of an antibody, wherein these domains are present in a single polypeptide chain. Generally, the scFv polypeptide further comprises a polypeptide linker between the VH and VL domains that enables the scFv to form the desired structure for antigen binding.

A “Fv” fragment is the smallest antibody fragment that retains an intact antigen binding site. A “dsFv” is a disulfide stabilized Fv. A “F (ab′) 2” fragment, is a bivalent fragment comprising two Fab fragments linked by a disulfide bridge at the hinge region. A “Fab” fragment is an antigen-binding fragment, containing complete light chains paired with the VH and CH1 domains of the heavy chain. A “Fab′” fragment is a reduced F (ab′) 2 fragment.

A “single domain antibody” (DAB) is an antibody with only one (instead of two) protein chain derived from only one of the domains of the antibody structure. DABs exploit the finding that, for some antibodies, half of the antibody molecule binds to its target antigen almost as well as the whole molecule (Davies et al. (1996) Protein Eng. 9: 531-537.

“Diabodies” are bivalent or bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (Holliger et al. (1993) Proc. Natl. Acad. Sci. USA, 90, 6444-6448).

In another embodiment the molecule of the invention is a polyclonal or monoclonal antibody, in another embodiment the antibody is a human or humanized antibody. In still another embodiment the antibody may include as light chain one or two scFv antibody fragment according to the invention. In still another embodiment the antibody is a anti-human PVR binding antibody, which may be selected from modified immunoglobulins, for example chemically or recombinantly produced antibodies or humanized antibodies, site directed mutagenized antibodies, which retain substantially the same affinity for PVR.

In another embodiment the polypeptide of the invention is a human antibody selected from the group consisting of IgA, IgD, IgE, IgG, and IgM, in particular IgG and IgM, more particularly IgG1, IgG2a, IgG2b, IgG3, IgG4.

By using a suitable molecule of the invention, particularly the antibody fragment or antibody, it is further possible to produce anti-idiotypic antibodies according to the invention. Such anti-idiotypic antibodies can be produced using well-known hybridoma techniques (Kohler et al. (1975) Nature, 256: 495). An anti-idiotypic antibody is an antibody, which recognizes unique determinants present on another antibody. These determinants are located in the hypervariable region of the antibody. It is this region, which binds to a given epitope and, thus, is responsible for the specificity of the antibody. An anti-idiotypic antibody can be prepared by immunizing an animal with the polypeptide, particularly the antibody fragment or antibody, of interest. The immunized animal will recognize and respond to the idiotypic determinants of the immunizing antibody and produce an antibody to these idiotypic determinants. By using anti-idiotypic antibodies, it is possible to identify other hybridomas expressing monoclonal antibodies having the same epitopic specificity. The anti-idiotypic antibodies according to the present invention can be used to screen antibodies and verify whether the antibody has the same binding specificity and functionality as a molecule of the invention.

It is also possible to use the anti-idiotype technology to produce monoclonal antibodies, which mimic an epitope. For example, an anti-idiotypic monoclonal antibody made to a first monoclonal antibody will have a binding domain in the hypervariable region, which is the “image” of the epitope bound by the first antibody. Thus, the anti-idiotypic monoclonal antibody can be used for immunization, since the anti-idiotype monoclonal antibody binding domain effectively acts as an antigen.

In another embodiment, the molecule of the invention can be covalently or non-covalently conjugated and/or coupled to or with, respectively, another protein, a solid matrix (e.g. like a bead), with itself to form multimers, a cytotoxic agent further enhancing the toxicity to targeted cells, a cytostatic agent, a prodrug, or an effector molecule, which is able to modify the cell expressing PVR or to recruit immune cells.

All these conjugates are “bioconjugates” according to the invention.

The term “bioconjugate” according to the present invention comprises the molecule of the invention, particularly the antibody fragment or antibody of the invention, together with one or more cytotoxic moieties, which are made using a variety of bifunctional protein coupling agents. Some examples of such reagents are N-succinimidyl 3-(2-pyridyldithio)-propionate (SPDP), bifunctional derivatives of imidoesters such a dimethyl adipimidate HCl, active esters such as disuccinimidyl suberate, aldehydes such as glutaraldehyde, bisazido compounds such as his (R-azidobenzoyl) hexanediamine, bisdiazonium derivatives such as bis-(R-diazoniumbenzoyl)ethylenediamine, diisocyanates such as tolylene 2,6-diisocyanate, and bis-activated fluorine compounds such as 1,5-difluoro-2,4-dinitrobenzene. Methods useful for the production of bioconjugates are know by someone skilled in the art and described in detail in March's Advanced Organic Chemistry: Reactions, Mechanisms and Structure, 5th Edition, Wiley-Interscience; or Bioconjugate Techniques, Ed. Greg Hermanson, Academic Press.

A list of preferred cytotoxic agents to link to a bioconjugate of the present invention includes, but is not limited to, daunorubicin, taxol, adriamycin, methotrexate, 5 FU, vinblastin, actinomycin D, etoposide, cisplatin, doxorubicin, genistein, andribosome inhibitors (e.g. trichosantin), or various bacterialtoxins (e.g. Pseudomonas exotoxin; Staphylococcus aureus protein A).

In one embodiment such molecules are molecules or oligonucleotides, which modulate the PVR function by inhibiting gene expression of PVR, for example antisense oligonucleotides or interfering double stranded RNAs (siRNAs) or vectors that lead to the cellular production of such siRNAs. Additionally, such molecules bind specifically to already expressed PVR and inhibit PVR function related to adhesiveness, invasiveness and/or metastatic potential of cells.

Antisense oligonucleotides are well known in the art as means to reduce gene expression of a specific gene (see, for example, Probst J. C. (2000) Methods 22 (3): 271-281; Heasman J. (2002) Dev. Biol. 243 (2): 209-214; Castanotto et al. (2000) Methods Enzymol. 313: 401-420 and Jen and Gewirtz (2000) Stem Cells. 18 (5): 307-319). Recently it has been found that short, double stranded RNAs potently and specifically inhibit expression of an mRNA with a complementary exonic sequence. This phenomenon has been termed RNA interference (RNAi). These double stranded RNAs have been termed siRNAs, for small interfering RNAs, have a paired region of at least 18, preferably at least 19 nucleotides (this paired, double stranded region targets them against an mRNA with a complementary exonic sequence), and have a length of 20 nucleotides to 25 nucleotides, preferably of 21 nucleotides to 23 nucleotides (Elbashir et al. (2001) Nature 411, 428-429). These siRNAs can be synthesized chemically by methods well known in the art and are commercially available (see e.g. suppliers given in Elbashir et al. above and also in Elbashir et al., (2002) Methods 26, 199-213) or by T7 transcription off a suitable DNA template (see Yu et al. (2002) PNAS 99, 6047-6052). They can be delivered to a wide range of cell types, in which inhibition of gene expression of a certain gene is desired, by, e.g., synthesizing the two RNA strands, <BR> annealing them and transfecting them (see Elbashir et al. and Yu et al., above). Detailed protocols for the application of siRNAs are described in Elbashir et al., (2002) Methods 26, 199-213.

Another way of effecting RNA interference is to transfect a plasmid that leads to the cellular production of siRNAs or siRNA like hairpin RNAs, which are also functional in RNAi. This can be done by plasmids which carry both, the sense and the antisense strand of the siRNA under the control of a mammalian, preferrably human or mouse, U6 promotor, which leads to the transcription of both strands in a transfected cell, some of which anneal to form functional double stranded siRNAs within the transfected cell (Miyagishi and Taira (2002) Nature Biotech. 19, 497-500). Alternatively, the RNA species transcribed from the U6 promotor can be siRNA-like hairpin RNAs which consist of a 19-base pair siRNA stem with the two strands joined by a structured loop and a U1 4 3′overhang at the end of the antisense strand (Paul et al. (2002) Nature Biotech. 19, 505-508). Alternatively the RNA species transcribed from the U6 promotor can be short hairpin RNAs, which are transcribed as small temporal RNAs, short hairpin precursors of about 70 nucleotides, and processed into active siRNAs within the cell in which they are transcribed (Paddison et al. (2002) Genes and Development 16, 948-958). All these methods based on transfection of plasmids have in common that they lead to the cellular production of siRNAs, which leads to the inhibition of the gene with an exonic region complementary to the at least 18 nucleotide long base paired region of the siRNA. It is clear that also future ways to affect the cellular presence of siRNAs will lead to the desired effect of inhibiting PVR function and are within the scope of the invention.

In another embodiment the molecule of the invention is labeled with a detectable label.

The terms “label” or “labeled” refers to a detectable marker or the incorporation of such, <BR><BR> respectively, e.g., by incorporation of a fluorophore-, chromophore- or radio-labeled amino acid or attachment of a fluorophore-, chromophore- or radiolabel to a polypeptide or attachment of moieties that can be detected by a labeled second molecule containing a fluorescent marker or enzymatic activity that can be detected by an optical or a colorimetric method. An example for such a two-step detection system is the well-known biotin-avidin system. Various methods of labeling polypeptides and glycoproteins are known in the art and may be used (Lobl et al. (1988) Anal. Biochem., 170, 502-511). Particularly, examples for detectable labels according to the present invention are radioisotopes, chromophores, fluorophores, enzymes or radioisotopes.

According to another embodiment the molecule of the invention is labeled and/or chemically modified with an inducible label to increase its biological activity. The induction of the chemical label can be activated by temperature rise, laser light induction or any other high energetic wave. According to one further embodiment of the present invention the molecules are chemically labeled with e.g. one or more chromphoric agent as described above to increase the biological activity of a ligand in combination with Chromophore-Assisted Laser Inactivation (CALI).

The principle of CALI is based on the local initiation of a photochemical reaction that leads to the generation of short-lived reactive species, which in turn selectively modify the target molecule and cause its functional inactivation. Highly specific but non-inhibitory ligands (e.g. antibodies, antibody fragments, small molecules) are labeled with a suitable chromophore (e.g. malachite green, fluorescein, methylene blue, eosin). After complex formation between the target molecule (e.g. proteins) and the ligand, the complex is irradiated with light (laser or visible light) of an appropriate wavelength to excite the chromophore. The excitation triggers a photochemical reaction that initiates the generation of short-lived reactive species (e.g. hydroxyl radicals or highly reactive oxygen species). These reactive species modify the protein within a small radius around their site of generation. The distance that a reactive species can travel is very short due to its short lifetime. Therefore, the modifications of amino acid residues within the protein occur in close proximity to the binding site of the ligand. The damaging effect is restricted to a radius of 15-40 A, which is well below the average distance of two proteins within a cell, which is at about 80 A, (assuming an average cytosolic protein concentration of 300 mg/ml and an average protein size of 50 kDa) ensuring a high spatial resolution of the process. In cases where the binding site of the ligand is close or within an important functional domain of the protein, the induced modifications lead to permanent inactivation of the protein. The functional inactivation of the protein is measured in an appropriate readout assay and evaluated in the context of disease relevant physiological functions like cell invasion, cell adhesion cell signaling or apoptosis. Therefore, these ligands can be used to modulate the action of inhibitory ligands.

In one embodiment the modified polypeptide or the bioconjugate of the invention binding to human PVR, reduces the adhesiveness and/or invasiveness of human cancer cells by 20-60%, or preferably by 30-55%, 40-50% or even at least 60%, when tested in an adhesion assay or an invasion assay (see Examples).

As a further example, a modified antibody against a given protein often can selectively inhibit the function of that particular protein after CALI, even if this antibody did not show an inhibiting function without CALI. In cases where the binding site of the ligand is close or within an important functional domain of the protein, these induced modifications lead to permanent inactivation of the protein. The functional inactivation of the protein is measured in an appropriate readout assay and evaluated in the context of disease relevant physiological functions like cell invasion, cell adhesion, cell signaling or apoptosis. It was shown by the inventors that CALI is able to convert specific but non-inhibitory ligands into blocking reagents (see, Examples).

According to a further embodiment of the invention cells expressing CD155, PVR or any derivative thereof are preferably proliferating cells, which are part of or involved in a proliferative disorder or disease. For example such cells are cancer cells, cells derived from a primary or metastatic tumor or from micro metastases. Furthermore, proliferative cells according to the invention are cells having a metastatic potential, e.g. cells having an increased readiness to traffick to and invade into distant tissue. In still another embodiment according to the present invention the cells expressing PVR, CD 155 or any of their derivatives originate from or derive from naturally occurring tumors or cancers.

A “cell originating from or deriving from a naturally occurring cancer” as used herein is a cell that has not been transfected, transduced or otherwise genetically engineered in the laboratory. Such a cell does not comprise any artificial DNA sequences, e.g. of vectors or DNA sequences being found only in other species, but not usually in the species from which the naturally occurring cancer cell was derived. However, a naturally occurring cancer cell may comprise sequences that are not usually found in the species from wherefrom it was derived, if those sequences have arisen due to the processes of mutation, viral infection and/or selection that took place within the individual from which the naturally occurring cancer cell was derived, and/or obtained during continued culture of the naturally occurring cancer cell.

“Metastatic tumors” as used herein includes both tumors at the primary site capable of metastasizing and metastasized tumors at a secondary site. Such metastatic tumors can be of any organ or tissue origin, like brain, central nervous system, lungs, stomach, lower intestine, liver, breast, prostate, kidneys or the pancreas, etc.

“Metastatic potential” as used herein is the ability of a tumor cell to form a new tumor at a site distant from the primary tumor of which the tumor cell was derived (a metastase). Metastatic potential can be measured by injecting, e.g. 1×106, cells into the lateral tail vein of athymic nude mice and determining the number of tumor nodules in the lung, e.g. 2 months post injection, e.g. as described in the section “Tumor cell injections” on page 2346 of Huang et al (1996) Oncogene 13, 2339-2347, or the sections “Animals and production of tumors” and “Histochemical analysis for calcified matrix” on page 1882 of Radinsky et al. (1994) Oncogene 9: 1877-1883. A cell line to produce more than 3, preferably more than 8, more preferably more than 20 tumor nodules in the lung in this assay is considered metastatic.

A “micro-metastase” is an accumulation of tumor cells with a size smaller than 2 mm, which can usually only be detected by histological methods.

According to a further embodiment the proliferating cells according to the present invention are derived from selected cancer cell types such as listed below.

Selected “cancer cell-types” as preferably screened according to the methods of the invention as well as preferably treated according to the invention are selected from the group comprising astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, melanoma, cholangiocarcinoma, synovioma, mesothelioma, Ewing's tumor, rhabdomyosarcoma, coloncarcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bileduct carcinoma, choriocarcinoma, seminoma, embryonal carcinoma, Wilms' tumor, testicular tumor, prostate, breast, medulloblastoma, craniopharyngioma, ependymoma, pinealoma, hemangioblastoma, acoustic neurorna, oligodendroglioma, meningioma, neuroblastoma, retinoblastoma, leukemia, multiplemyelorna, Waldenstrom's macroglobulinemia, and heavy and light chain disease, and adenocarcinomas of the uterine cervix, uterine and ovarian epithelial carcinomas, transitional cell carcinoma of the bladder, B and T cell lymphomas (nodular and diffuse) plasmacytoma, acute and chronic leukemias, soft tissue sarcomas, leiomyosarcomas or any other tumor cell with high PVR expression.

According to a further embodiment and as exemplatory result of one experimental set up, as described herein and in the examples, a scFv having the amino acid sequence SEQ ID No.: 3, SEQ ID No.: 4, SEQ ID No.: 69 or SEQ ID No.: 70 or an CDR having the amino acid sequence LWLRRD (SEQ ID No.: 1) or WTGDFDY (SEQ ID No.: 2) and specifically binding to PVR as well as modulating its function was isolated.

In another embodiment of the invention the molecule, polypeptide and/or the bioconjugate of the invention are used for identifying additional molecules that specifically bind human PVR in screening assays. These methods entail contacting a reference anti-PVR molecule or antibody fragment with a target species comprising the PVR domain in the presence of a putative competitor test-binding agent. This step of contacting is conducted under conditions suitable for complex formation between the reference antibody fragment and the target species in the absence of the test-binding agent. Complex formation between the reference antibody fragment and the target species in the presence of the test-binding agent is detected as an indicator of specific binding activity of the test-binding agent to PVR. This screening method is useful for high throughput screening of, e.g., other antibody libraries or antibody fragment libraries, antisense oligonucleotide libraries or peptide and small molecule libraries to identify and characterize additional molecules binding specifically to PVR. Competition is determined by an assay in which the antibody fragment, or other binding agent under test substantially inhibits specific binding of the reference antibody fragment to the target species containing the PVR domain. This can be determined for example by measuring binding of the reference antibody fragment to a target species comprising PVR domain in the presence and absence of a putative competitor, i.e. a molecule binding specifically to PVR under conditions suitable for complex formation. Numerous types of competitive binding assays are known and routinely practicable within the invention, as described for example in U.S. Pat. No. 4,376,110. Typically, such assays involve the use of a target species containing the PVR domain (e.g., purified PVR or a cell line expressing the PVR antigen), an unlabeled molecule binding specifically to PVR, and a labeled reference antibody fragment or other binding agent. Competitive inhibition is measured by determining the amount of label bound to the target species in the presence of the molecule binding specifically to PVR. Usually the molecule binding specifically to PVR is present in excess. Molecules binding specifically to PVR identified by these competition assays (“competitive binding agents”) include antibodies, antibody fragments, peptides, antisense oligonucleotides, small molecules and other binding agents that bind to an epitope or binding site bound by the reference antibody fragment, as well as a molecule binding specifically to PVR that bind to an epitope or binding site sufficiently proximal to an epitope bound by the reference antibody fragment.

Preferably, competitive binding agents of the invention will, when present in excess, inhibit specific binding of a reference antibody fragment to a selected target species by at least 10%, preferably by at least 25%, more preferably by at least 50%, and even more preferably by at least 75%-90% or greater. In addition to a polypeptide of the invention, particularly a modified antibody fragment or a modified antibody of the invention, natural or artificial ligands, peptides, anti-sense, or other small molecules capable of specifically targeting human PVR may be employed.

The present invention further relates to a method to amplify the molecule of the invention, including an antibody fragment, more particularly a scFv, dsFv, Fv, single domain antibody or diabody of the invention by recombinant techniques. These techniques are well known in the art (Skerra et al. (1993), Curr. Opin. Immunol. 5, 256-62; Chadd et al. (2001), Curr. Opin. Biotechnol. 12, 188-94).

For example, nucleic acid sequences encoding a polypeptide of the invention, <BR><BR> particularly an antibody fragment or an antibody (e.g., a gene encoding SEQ ID No.: 1 to 4) can be isolated and cloned into one or more expression vectors, and the vector can be transformed into a suitable host cell line for expression of a recombinant polypeptide of the invention. Expression of the gene encoding the polypeptide of the invention provides for increased yield of the polypeptide, and also allows for routine modification of the polypeptide by introducing amino acid substitutions, deletions, additions and other modifications, for example humanizing modifications (Rapley (1995) Mol. Bioteclmol. 3: 139-154) in both the variable and constant regions of the antibody fragment or of the antibody of the invention without critical loss of binding specificity or PVR blocking function (Skerra et al. (1993) Curr. Opin. Immunol. 5, 256-262).

Accordingly, in a further embodiment the invention comprises the nucleic acid sequences derived from isolated molecules according to the invention. In still a further embodiment the invention provides the nucleic acid sequence according SEQ ID No.: 5, SEQ ID No.: 6, SEQ ID No.: 71, or SEQ ID No.: 72 as well as homologous DNA sequences or DNA sequences having a degenerated code but still translating into an amino acid sequences having a substantial identity with SEQ ID No.: 1 to 4 or SEQ ID No.: 69 or SEQ ID No.: 70. “Substantial amino acid sequence identity” as used herein means that at least 70%, preferably at least 75%, 80%, 85%, 90%, more preferably all but 5, still more preferably all but 3 and even more preferably all but 1 of the amino acids of two aligned amino acid sequences, particularly of aligned CDRs, are identical.

The present invention further relates in another embodiment to a procaryotic or eucaryotic expression vector comprising a nucleic acid of the invention. The vector is for example a plasmid, a phagemid, or a cosmid. Such expression vectors comprise at least one promotor, at least one signal for translation initiation, at least one nucleic acid sequence of the invention and—in the case of procaryotic expression vectors—a signal for translation termination, while in the case of eucaryotic expression vectors preferably additional signals for transcriptional termination and for polyadenylation.

Examples for prokaryotic expression vectors are, for expression in Escherichia coli, e.g. expression vectors based on promoters recognized by T7 RNA polymerase, as described in U.S. Pat. No. 4,952,496, for eucaryotic expression vectors for expression in Saccharomyces cerevisiae, e.g., the vectors p426Met25 or 526GAL1 (Mummberg et al. (1994) Nucl. <BR><P> Acids Res., 22, 5767-5768), for the expression in insect cells, e.g., Baculovirus-vectors as e.g. described in EP-B1-0 127 839 or EP-B1-0 549 721, and for the expression in <BR><BR> mammalian cells, e.g., the vectors Rc/CMV and Rc/RSV or SV40-vectors, which are commonly known and commercially available.

The molecular biological methods for the amplification of these expression vectors, as well as the methods of transfecting host cells and culturing such transfected host cells as well as the conditions for producing and obtaining the polypeptides of the invention from said transformed host cells are well known to the skilled person. For example the nucleic acid molecule of the invention can be cloned in a suitable fashion into the <BR><BR> expression vectors according to Sambrook et al., (1989) (“Molecular cloning: a laboratory manual” Second edition, Cold Spring Harbor Laboratory Press).

The present invention further relates to a host cell comprising the nucleic acid of the invention and/or the vector of the invention, particularly wherein the host cell is a microorganism like yeast or other fungi, like Escherichia coli, Bacillus subtilis or other bacteria. The host cell can also be a cell of higher eucaryotic origin, like an insect cell, preferably a virus infected insect cell, more preferably a baculovirus infected insect cell, or like a mammalian cell like HeLa, COS, MDCK 293-EBNA1, NSO or a hybridoma cell.

The present invention further provides a method for the production or reproduction of a polypeptide of the invention, particularly an antibody fragment of the invention, comprising culturing a microorganism transformed with a recombinant vector comprising DNA according to the present invention and recovering a relevant polypeptide of the invention, particularly an antibody fragment of the invention or a fusion protein containing it, from the medium.

According to a further embodiment of the invention the expression of a PVR on the surface of a cell can be detected in an assay using a molecule, bioconjugate or polypeptide, antibody or antibody fragment comprising any of the sequences as listed in the sequence list under SEQ ID No.: 1 to 4 or SEQ ID No.: 69 or SEQ ID No.: 70. For this assay a sample is taken from the subject, e.g. a biopsy specimen taken from tissue suspected of having a proliferative disease. Generally, the sample is treated in well-known manner before an assay is performed. Assays, which can be employed, include ELISA, RIA, EIA, Western Blot analysis, immuno-histological staining and the like. Depending upon the assay used, the antigens or the antibodies can be labeled by an enzyme, a fluorophore or a radioisotope (See, e.g. Coligan et al. (1994) Current <BR><BR> Protocols in Immunology, John Wiley &amp; Sons Inc., New York, N.Y.; and Frye et al. (1987) Oncogene 4, 1153-1157). This assay is particularly useful for identifying patients, which can be susceptible to a treatment with PVR modulating agents, since the cells derived from their primary tumor or their metastasis show a PVR expression.

The present invention further provides a method to modulate and determine the adhesiveness of a naturally occurring cancer cell expressing PVR. This method measures the adhesiveness of a target, e.g. a cell to something else, e.g. another cell, a virus, a complex biological mixture, e.g. extracellular matrix or basal lamina. The method comprises the steps of:
a. contacting the cells with a molecule modulating PVR function;
b. contacting the cancer cell with a layer of ECM proteins, under conditions suitable for the growth of said cancer cells;
c. analyzing the adhesion of said cancer cells to the layer of ECM proteins;
d. optionally, activating an inducible label attached to the molecule and performing step c) again; and
e. determining the percentage of reattachment of cells in step c) and, optionally, step d) in comparison with untreated cells.

The term “layer of ECM proteins” as used herein is understood to be a semi-dry layer of a protein solution, which allows cultivation of cancer cells in contact with the layer and allows attachment of invasive cancer cells to said layer. The thickness of said “layer of ECM proteins” is between 0.1 mm to 1 mm, preferably 0.3 mm thick. Examples for ECM proteins are substances of the extracellular matrix. Particularly ECM proteins are selected from the group consisting of the proteins collagens, entactin, nidogen, vitronectin, fibronectin and laminins. More particularly ECM proteins are selected from collagen S type I, collagen type IV, fibronectin and laminin.

In a preferred embodiment the interfering molecule of step a) is a molecule that specifically binds to an extracellular epitope of PVR, particularly a modified molecule of the invention, more particularly a modified antibody or a modified antibody fragment of the invention, still more particularly a modified antibody fragment, even more particularly a modified scFv, dsFv, Fv, single domain antibody or diabody.

In another embodiment step a) of said method comprises the use of an antisense oligonucleotide against PVR, siRNA or siRNA-like hairpin RNA against PVR or a vector leading to cellular presence of siRNA against PVR or siRNA-like hairpin RNA against PVR. Particularly preferred is the use of siRNA or siRNA-like hairpin RNA against PVR or a vector leading to cellular presence of siRNAs against PVR or siRNA-like hairpin RNA against PVR.

In this particular embodiment of the invention the antisense oligonucleotide molecule inhibits gene expression of PVR. Preferably, the molecule inhibiting gene expression of PVR can be an antisense oligonucleotide targeted against PVR, an interfering double stranded RNA, commonly known as siRNA (small interfering RNA) targeted against PVR, a siRNA-like hairpin RNAs targeted against PVR function as inhibitors of gene expression, or a vector leading to cellular presence of siRNA targeted against PVR or siRNA-like hairpin RNAs targeted against PVR.

In another preferred embodiment step d) is introduced to the method to activate an inducible label attached to the molecule according to the invention that specifically binds to PVR. If for examples the attached labels are chromophores the an activation via CALI, as described before, may destroy a close-by active domain of the PVR and thereby increase the modulation and/or inhibition of the PVR functionality. A molecule, which previously showed only moderate capability to modulate PVR functionality, e.g., had only neutralizing capacity or even was only binding to the PVR, can thereby become a highly potential modulator or inhibitor of the PVR function (s). The described method allows to quantify the modulation or inhibition of the adhesiveness of cancer cells in comparison with cells bound by non-activated molecules and/or untreated cells.

The adhesive interaction between tumor cells and host cells or the extracellular matrix has been considered as an essential step in the process of metastasis formation. Interference in the adhesive potential of tumor cells with adhesion-blocking agents is therefore a promising approach for preventing metastasis. It is an achievement of the present invention not only to provide molecules binding to the PVR, with maybe neutralizing capacity as described in Lange et al. ((2000) Virology, 285, 218-227), but due to the sophisticated and combined screening, selection and, optional, induction method to identify, isolate and provide molecules which essentially modulate or destroy PVR functions on cells deriving from a proliferative disorder or disease and/or from a naturally occurring cancer. A preferred modulation is an inhibition of a tumor related adhesion behavior, further preferred as modulation is a prevention of a tumor related adhesion behavior by the destruction of active domains of the PVR.

However, an activation or increase of the adhesion behavior e.g. an increase of adhesion behavior to selected immune cells, like e.g. natural killer cells, or to synthetic elements, which could e.g. extract such tumor cells in a blood dialysis, is also provided. In summary, the invention provides molecules or modified or labeled molecules, which actively prevent by binding to or destroying off active domains the tumor related PVR function (s), namely e.g. the adhesive behavior of cancer cells.

The migration of tumor cells into tissue is also an important step in metastasis. The processes of invasion can be studied in the transendothelial model (See, Woodward et al. (2002) Invest Opthalmol Vis Sci 43, 1708-14 and Vachula et al. (1992) Invasion Metastasis 12, 66-81). A similar transendothelial model provides a further useful in vitro assay for the identification of molecules as described above and of screening of molecules modulating or inhibiting the PVR mediated invasion process.

The present invention therefore further provides a method to modulate and determine the invasiveness of a naturally occurring cancer cell expressing PVR. This method comprises the steps of:
a. contacting the cells with a molecule modulating PVR function
b. contacting the cancer cell with a gel-like matrix, under conditions suitable for the growth of said cancer cells; and
c. analyzing the migration of said cancer cells through the gel-forming matrix;
d. optionally, activating an inducible label attached to the molecule and performing step c) again; and
e. determining the percentage of migration of cells in step c) and, optionally, step d) in comparison with untreated cells.

The term “gel-like matrix” as used herein is understood to be a semi-solid substance with a water content of at least 90%, which allows cultivation of cancer cells in contact with the matrix and allows migration of invasive cancer cells through a slab of said “gel-like matrix” of 0.1 mm to 1 mm, preferably 0.3 mm thickness, but not migration of non-invasive cells. Examples for such a “gel-like matrix” are substances resembling the extracellular matrix in protein and carbohydrate composition, particularly the commercially available “Matrigel”. Particularly the “gel-like matrix” comprises one of the proteins selected from the group consisting of the proteins collagen type IV, fibronectin and laminin. More particularly the gel-like matrix comprises the proteins collagen type IV, fibronectin and laminin. More preferable the gel-like matrix comprises the proteins collagen type IV, laminin, entactin, nidogen and heparan sulfate proteoglycans or collagen type IV, fibronectin, laminin, nidogen, entactin, and vitronectin.

In a preferred embodiment the interfering molecule of step a) is a molecule that specifically binds to an extracellular epitope of PVR, particularly a modified polypeptide of the invention, more particularly a modified antibody or a modified antibody fragment of the invention, still more particularly a modified antibody fragment, even more particularly a modified scFv, dsFv, Fv, single domain antibody or diabody.

In another preferred embodiment step a) of said method comprises the use of an antisense oligonucleotide against PVR, siRNA or siRNA-like hairpin RNA against PVR or a vector leading to cellular presence of siRNA against PVR or siRNA-like hairpin RNA against PVR as described above.

In another preferred embodiment step d) is introduced to the method to activate an inducible label attached to the molecule according to the invention that specifically binds to PVR. If, for example, the attached labels are chromophores the activation via CALI, as described later, may destroy a close-by active domain of the PVR and thereby increase the modulation and/or inhibition of the PVR functionality. A molecule, which previously showed only moderate capability to modulate PVR functionality, e.g. had only neutralizing capacity for one but not another function or even was only binding to the PVR, can thereby become a highly potential modulator or inhibitor of the PVR function (s). The described method allows a quantification of the modulation or inhibition of the invasiveness of cancer cells in comparison with cells bound by non-activated molecules and/or untreated cells. Molecules according to the invention that modulate or inhibit the invasive behavior of cancer cells by more than 20%, more that 40%, more than 60% or even more than 80% are preferred.

The described method, furthermore, allows the selection of the most suitable molecule according to the invention for an individual patient to avoid thereby any individual mismatches, which could interfere with a therapeutic approach.

An angiogenesis assay measures the ability of a cell to induce blood vessel formation, a process, which usually accompanies the growth of malignant tissue. An angiogenesis assay can be performed as described earlier in Kanda et al (2002) J. Natl. Cancer Inst. 94, 1311-9.

The described assays are on one hand side involved in the method to identify suitable molecules according to the invention, which beside binding to the PVR also modulate its function. However, these methods are also highly useful isolated from the method of identifying molecules according to the invention, since they provide a highly selective tool to screen the cancer cells derived from a patient (e.g. by a biopsy) to select suitable molecules according to the invention, which react best with the individual PVR of a patient and thereby show the most effective modulation of the patient derived cancer cells. Preferably, the molecule identified in an in vitro assay for a subsequent treatment should inhibit the adhesion, trafficking or invasion behavior of the patient derived tumor cells by about 20, preferably 40%, 50%, 60%, 80% or up to 90%. The subsequent treatment can thereby be specifically tailored and, thus, individual deviations in the receptor binding activities can be avoided.

In another embodiment, the present invention relates to a pharmaceutical composition comprising effective amounts of at least one, but not limited to one molecule according to the invention, particularly a small chemical compound, an antibody, an antibody fragment or bioconjugate modulating the PVR functions or at least one of the molecules inhibiting gene expression of PVR, more particularly wherein the molecule is an antisense oligonucleotide against PVR, an interfering dsRNA (siRNA) or siRNA-like hairpin RNA against PVR or a vector leading to cellular presence of siRNA against PVR or siRNA-like hairpin RNA against PVR, in combination with a pharmaceutically acceptable carrier and/or a diluent. The molecules inhibiting PVR function may also be used as the single effective compound as a medicament or a therapeutic composition.

“Therapeutically effective amounts” are amounts which eliminate or reduce the patient's tumor burden, or which prevent, delay or inhibit metastasis. The dosage will depend on many parameters, including the nature of the tumor, patient history, patient condition, the possible co-use of cytotoxic agents, and methods of administration. Methods of administration include injection (e.g. parenteral, subcutaneous, intravenous, <BR><BR> intraperitoreal, etc.), for which the molecule inhibiting PVR function is provided in a nontoxic pharmaceutically acceptable carrier.

“Pharmaceutically acceptable carriers” are, in general, suitable carriers and diluents selected so as not to significantly impair biological activity of the binding agent (e.g. binding specificity, affinity or stability), such as water, saline, Ringer's solution, dextrose solution, 5% human serum albumin, fixed oils, ethyloleate, or liposomes. Acceptable carriers may include biocompatible, inert or bio-absorbable salts, buffering agents, oligo- or polysaccharides, polymers, viscoelastic compound such as hyaluronic acid, viscosity-improving agents, preservatives, and the like. In addition, the pharmaceutical composition or formulation may also include other carriers, adjuvants, or nontoxic, non-therapeutic, non-immunogenic stabilizers, diluents and the like. Typical dosages may range from about 0.01 to about 20 mg/kg, or more particularly from about 1 to about 10 mg/kg.

The pharmaceutical composition can be used for the treatment of conditions related to the over-expression or ectopic expression of human PVR, especially the treatment of tumors, tumors with metastatic potential and/or micro-metastases, especially of metastatic tumors derived from the group selected of cancer cell-types as described above.

In another embodiment, the invention relates to a method of treating or preventing any proliferative disorder or disease, primary or secondary tumors as well as metastasis in a patient comprising the administration of a molecule inhibiting PVR function, in a pharmaceutical acceptable composition in an amount effective to treat, i.e. inhibit, delay or prevent metastasis of PVR mediated adhesion and/or invasion, particularly wherein the molecule inhibits gene expression of PVR, more particularly wherein the molecule is an antisense oligonucleotide against PVR, an interfering dsRNA (siRNA) or siRNA-like hairpin RNA against PVR or a vector leading to cellular presence of siRNA against PVR or siRNA-like hairpin RNA against PVR. Alternatively the molecule inhibiting PVR function can particularly be a molecule which can bind to the extracellular region of PVR, which can be identified by the method of identifying a ligand binding specifically to the extracellular region of PVR, more particularly wherein the molecule is a small chemical compound or an antibody or an antibody fragment or a modified polypeptide of the invention or a bioconjugate of the invention, still more preferably wherein the molecule is a modified scFv of the invention or a modified antibody derived from such a scFv of the invention. Furthermore, the molecule can be labeled by chromophores, fluorophores, enzymes or radioisotopes, which can be induced or activated in vivo by e.g. the administration of a chemical inducer or the exposition to a photochemical activator. The method of treating or preventing a proliferative disorder, cancer, metastatic tumors, micro-metastases and/or metastasis can be effective to reduce or inhibit the adhesion and/or invasion of naturally occurring cancer cells in particular cancer cells derived from metastatic tumors derived from the group of selected cancer cell-types as described above, since it was shown that blocking PVR function inhibits adhesiveness and/or invasiveness of cancer cells derived from said selected group. In a preferred embodiment, treating of patients according to the present invention, which have naturally occurring cancer cells expressing the PVR with one or more modified antibody fragments causes or leads to a reduction or inhibition of the adhesive and/or invasive ability of human sarcoma cells.

“Treating metastatic tumors” or “treating micro-metastases”, as used herein means that cells having a metastatic potential or that metastases of the tumor are stabilized, prevented, delayed, or inhibited by the molecule of the invention, either as a single medicament or in combination with other medicaments. Stable disease or “No Change” (NC) is a description for the course of the disease with either no change of the metastases or a reduction of less than 50% or an increase of less than 25% over at least 4 weeks. Prevention can be, for example, that no new metastases are detected after the treatment is initiated. This can lead to a two- to three-fold median and/or a 5-year survival rate of treated patients compared with untreated patients. A delay can signify a period of at least 8 weeks, 3 months, 6 months or even one year in which no new metastases are detected after the treatment is initiated. Inhibition can mean that the average size or the total number of new metastases is at least 30%, 40%, 50%, 60%, 70%, 80% or even 90% lower in a group treated with the molecule of the invention in comparison with an untreated group. Number, size and prevalence of metastases can be detected by a skilled practitioner in the field of oncology following generally accepted practice and diagnostic procedures for the detection of metastases, for example as outlined in Harrisons Principles of Internal Medicine 15ted 2001 Me Graw Hill.

According to a further embodiment the method of treating according to the invention can be combined with various therapeutic methods. In this context a combination of the treatment with the term “therapeutic methods” means employing the method of treatment with molecules inhibiting PVR function combined with chemotherapy, surgery, and radiation therapy, depending on type of the tumor, patient condition, other health issues, and a variety of factors.

One embodiment of the invention is therefore the use of at least one molecule which can bind to the extracellular region of PVR, which can be identified be the method of identifying a ligand binding specifically to the extracellular region of PVR, more particularly wherein the molecule is a small chemical compound or an antibody or an antibody fragment or a modified polypeptide of the invention or a bioconjugate of the invention, still more preferably wherein the molecule is a modified scFv of the invention or a modified antibody derived from such a scFv of the invention, furthermore, a labeled molecule according to the invention, which can be induced or activated in vivo by the administration or application of an inducer for the manufacture of a medicament for the treatment or prevention of adhesion, invasion and/or any metastatic potential of naturally occurring cancer cells, wherein adhesiveness, invasiveness and/or the metastatic potential of said cancer cells is influenced by the PVR function.

The invention therefore provides a method for the production of a pharmaceutical composition or medicament comprising the step of incorporating the molecule or the ligand according to the present invention into a therapeutic, prophylactic or diagnostic composition. This can, e.g., be done by mixing the identified ligand or a modified ligand with a pharmaceutically acceptable carrier known in the art, wherein the ligand is present in an amount, which is therapeutically effective.

In another embodiment, the present invention encompasses a diagnostic kit. Such a kit comprises at least one bioconjugate and/or at least one labeled molecule or polypeptide of the invention and/or an antibody fragment or an antibody of the invention, or a labeled version of these, and consists additionally of the reagents and materials necessary to carry out a standard competition or sandwich assay. Said diagnostic kit may be used for the determination of the invasive potential of biological samples, in particular of certain cancer cell types. A kit will further typically comprise a container.

The following examples, including experiments conducted and results achieved, are provided for illustrative purposes only and are not to be construed as limiting upon the present invention.

### DESCRIPTION OF THE DRAWINGS

FIG. 1 shows the invasion of stained HT1080 cells through a Matrigel coated Sum filter. Fluorescence was quantified after six hours incubation at 37° C. Data presented are the mean of n=3 wells+/−SD.

FIG. 2a shows the inhibitory effect of scFv1 on the invasion of HT1080 cells. Invasion was determined in a chemotaxis assay with a Matrigel coated cell migration chamber. The invasion was determined after laser irradiation (with CALI). The invasion of HT1080 cells in the absence of any inhibitory molecule was used as a control (left bar). The molecule scFv1 inhibited the invasion of HT1080 cells by about 22% (p-value<0.05)

FIG. 2b shows the inhibitory effect of a commercial anti PVR antibody (D171), scFv3 and scFv4 on the invasion of HT1080 cells. Invasion was determined in a chemotaxis assay with a Matrigel coated cell migration chamber. The invasion was determined before and after light irradiation (black bars without CALI, gray bars with CALI). The invasion of HT1080 cells in the absence of any inhibitory molecule (0) was used as a control (left bars). The molecule scFv3 and scFv4 inhibited the invasion of HT1080 cells by about 30% (p-value<0.001).

FIG. 2c shows the inhibitory effect of scFv3 and scFv4 on the invasion of MDA-MD231 cells. Invasion was determined in a chemotaxis assay with a Matrigel coated cell migration chamber. The invasion was determined before and after light irradiation (black bars without CALI, gray bars with CALI). The invasion of HT1080 cells in the absence of any inhibitory molecule (0) was used as a control (left bars). scFv3 and scFv4 inhibited the invasion of MDA-MD231 cells by about 23% (p-value<0.001).

FIG. 2d shows the inhibitory effect of scFv3* and scFv4* on the migration of two different glioblastoma cell lines (U87MG cells=left set of bars, A172=right set of bars). Migration was determined before and after light irradiation (black bars without CALI, gray bars with CALI). The migration of cells in the absence of any inhibitory molecule (control) was used as a control (left bars in each set). The symbol “*” indicates that the respective scFv—as identified by its number as shown in FIG. 10 and FIG. 11—was cloned into an IgG format.

FIG. 3 shows the inhibitory effect of scFv1 and scFv2 on the adhesion of HT1080 cells to collagen S type 1. The adhesion was determined after laser irradiation (with CALI). The adhesion of HT1080 cells in the absence of any inhibitory molecule was used as a control (left bar). The molecule scFv1 inhibited the adhesion by about 56% and the molecule scFv2 by 42% (p-value<0.05).

FIG. 4 shows a result of FACS analysis. The binding activity of scFv1 and scFv2 to HT1080 cells (bold line) and to HS-27 cells (dotted line) as control is illustrated. Also the binding of scFv1 to PC3-cells, KHOS-cells and Liver cells is illustrated.

FIG. 5 shows the result of the immunoprecipitation experiments. scFv1 and scFv2 were incubated with lysates of HT1080 cells and of HS-27 cells as a control. The immuno-complexes were separated by SDS-PAGE and silver stained. The scFv band and also the PVR specific band are indicated.

FIG. 6 shows a MALDI-MS spectrum of the peptide mixture obtained from the band with an approximate size of 75 kDa immunoprecipitated with scFv 1. Two trypsin auto digestion peaks, indicated as T, were used for internal calibration. A total of 6 peaks, marked with asterisks, matched PVR (SwissProt, P15151), with a mass deviation of less than 10 ppm. The matched peptides cover 18% (74/417 residues) of the protein.

FIG. 7 shows the vector map of the scFv display vector pXP 10 and corresponding sequence (SEQ ID No.: 7).

FIG. 8 shows the vector map of the scFv expression vector pXP 14 and corresponding sequence (SEQ ID NO.: 8).

FIG. 9 shows the amino acid sequences (SEQ ID NO.: 3) and nucleotide sequence (SEQ ID NO.: 5) of scFv1.

FIG. 10 shows the amino acid sequences (SEQ ID NO.: 4) and nucleotide sequence (SEQ ID NO.: 6) of scFv2.

FIG. 11 shows the amino acid sequences (SEQ ID NO.: 69) and nucleotide sequence (SEQ ID NO.: 71) of scFv3.

FIG. 12 shows the amino acid sequences (SEQ ID NO.: 70) and nucleotide sequence (SEQ ID NO.: 72) of scFv4.

FIG. 13 shows the principle of Chromophore Assisted Laser Inactivation (CALI).

FIG. 14 shows the sequences of the primers SEQ ID No.: 9 to 52, 63 to 68.

### EXAMPLES

### Example 1

### Construction of an Immune Library

Two BALB/c mice were each immunized intradermally with 2×107 paraformaldehyde fixed HT1080 cells (human fibrosarcoma cell line; ATCC, CCL-121). Following the first immunization, the injections were repeated twice in a period of 39 days, the mice sacrificed and the spleens isolated and frozen in liquid nitrogen.

Total RNA was isolated using the RNeasy Midi Kit (QIAGEN #75142) as described by the manufacturer using half of each spleen preparation. The RNA concentration and purity was determined by a denaturing formaldehyde gel and photometric measurement. cDNA was synthesized using 8.9 llg of freshly prepared RNA and 10 pmol of a primer mix (IgG1-c (SEQ ID NO.: 63), IgG2a-c (SEQ ID NO.: 64), IgG2b-c (SEQ ID NO.: 65), IgG3-c (SEQ ID NO.: 66), VLL-c (SEQ ID NO.: 67), VLK-c <BR><BR> (SEQ ID NO.: 68)) using the Superscript II Kit (GibcoBRL Life Technologies #18064-014). These primers anneal to the RNA encoding the IgG heavy-chain (VH) genes and the light chain (VL) genes of the kappa and lambda family. VH genes were PCR amplified from 1 1 ll of cDNA using 36 individual combinations of 9 forward primers (M-VH1 (SEQ ID NO.: 9), M-VH2 (SEQ ID NO.: 10), M-VH3 (SEQ ID NO.: 11), M-VH4 (SEQ ID NO.: 12), M-VH5 (SEQ ID NO.: 13), M-VH6 (SEQ ID NO.: 14), M-VH7 (SEQ ID NO.: 15), M-VH8 (SEQ ID NO.: 16), M-VH9 (SEQ ID NO.: 17)) and 4 backward primers (M-JH1 (SEQ ID NO.: 18), M-JH2<BR> (SEQ ID NO.: 19), M-JH3 (SEQ ID NO.: 20), M-JH4 (SEQ ID NO.: 21)) without restriction sites. VL genes were PCR amplified with one primer mix (M-VK1 (SEQ ID NO.: 22), M-VK2 (SEQ ID NO.: 23), M-VK3 (SEQ ID NO.: 24), M-VK4 (SEQ ID NO.: 25), M-VL1 (SEQ ID NO.: 26), M-JK1 (SEQ ID NO.: 27), M-JK2<BR><BR> (SEQ ID NO.: 28), M-JK3 (SEQ ID NO.: 29), M-JL1 (SEQ ID NO.: 30)) without restriction sites. PCR products were gel-purified (QIAquick Gel Extraction Kit, #28706) and reamplified using individual combinations of 9 forward primers (MVH1 SfiI (SEQ ID NO.: 31), MVH2 SfiI (SEQ ID NO.: 32), MVH3 SfiI (SEQ ID NO.: 33), MVH4 SfiI (SEQ ID NO.: 34), MVH5 SfiI (SEQ ID NO.: 35), MVH6 SfiI (SEQ ID NO.: 36), MVH7 SfiI (SEQ ID NO.: 37), MVH8 SfiI (SEQ ID NO.: 38), <BR><BR> MVH9 SfiI (SEQ ID NO.: 39)) and 4 backward primers (M-JH1 SalI (SEQ ID NO.: 40), M-JH2 Sail (SEQ ID NO.: 41), M-JH3 Sail (SEQ ID NO.: 42), M-<BR><BR> JH4 SalI (SEQ ID NO.: 43)) with restriction sites for VH and one primer mix (M-VK1 ApaLI (SEQ ID NO.: 44), M-VK2 ApaLI (SEQ ID NO.: 45), M-VK3 ApaLI (SEQ ID NO.: 46), M-VK4 ApaLI (SEQ ID NO.: 47), M-VL1 ApaLI (SEQ ID NO.: 48), M-JK1 NotI (SEQ ID NO.: 49), M-JK2 NotI (SEQ ID NO.: 50), M-JK3 NotI (SEQ ID NO.: 51), M-JL1 NotI (SEQ ID NO.: 52)) with restriction sites for VL. PCR products were gel-purified (QIAquick Gel Extraction Kit, #28706) and cloned into the phage display vector pXP 10 (SEQ ID No.: 7) using the restriction sites Sf I/SalI for VH and ApaLIlNotI for VL. The ligation mix was transfected into E. coli TG-1 by electroporation resulting in a library size of 107 independent clones (phages) expressing different single chain antibody fragments (scFv).

### Example 2

### Selection of Tumor Cell Specific scFv (Selection on Fixed Cells)

Phages expressing scFv with high affinity to tumor cells were selected as follows: HT1080 cells were harvested with 0.05% EDTA, fixed with paraformaldehyde, diluted to 1×10 cells/ml in PBS and immobilized onto wells of a 96 well UV cross-link plate (Corning Costar). The wells of the UV cross-link plate were blocked with 5% Skim Milk Powder (#70166, Fluka) in PBS (MPBS). 1012 cfu (colony forming units) of phage library/106 cells were pre-blocked for 1 hour at 25° C. with MPBS and subsequently incubated for 1.5 hour at room temperature (RT) with the cells. The wells of the UV cross-link plate were washed six times with PBS+0.05% Tween-20 followed by six washes with PBS. Bound phage were eluted by the addition of 10 mM Glycine pH 2.2, and neutralized with 1 M Tris/HCl pH 7.4. Typically, between 103 and 106 cfu were eluted in the around of selection, thus the diversity of the enriched repertoire is decreased compared to the original repertoire. The eluate containing the enriched repertoire was amplified by infecting exponentially growing E. coli TG1. Phagemid containing E. coli were selected and propagated by overnight (o/n) growth at 30° C. on LB agar plates supplemented with 100 Rg/ml ampicillin and 1% glucose. Following this step, the enriched repertoire can either be amplified as a polyclonal pool and used for further rounds of selection in an iterative manner until convergence to desired properties is achieved or be spatially separated and screened on a single clone level. Phage particles for the next round of selection were produced by super-infecting exponentially growing cultures of the previous round of selection with helper phage VCS-M13 (Stratagene, La Jolla, Calif.) and growing the cultures overnight at 20° C. in 2×TY supplemented with 100 Ag/ml ampicillin (amp) and 50 Fg/ml kanamycin (kan). Selection ready phage were precipitated with 0.5 M NaCl/4% PEG-6000 from the cleared bacterial supernatant and re-suspended in PBS. One round of selection was performed, followed by screening on a single clone level as described in Example 3.

### Example 3

### Screening of scFv (Screening on Fixed Cells)

For screening, the genes encoding the selected scFv, contained in the phage display vector, were re-cloned to the expression vector pXP14 (SEQ ID No.: 8). This vector directs the expression of scFv in fusion with a Strep-tag and E-tag and does not contain a filamentous phage gene-3. Expression vector containing E. coli TG1 from single colonies were grown in individual wells of a micro titer plate so that each well contains only one scFv clone. The bacteria were grown at 30° C. in 2×TY supplemented with 100 Fg/ml ampicillin and 0.1% glucose in 96-well micro titer plates (#9297, TPP) until an OD600 of 0.7. Expression was induced with IPTG at a final concentration of 0.5 mM and continued at 25° C. overnight. Single chain Fv containing cleared lysates were prepared by addition of hen-egg lysozyme (#L-6876, Sigma) to a final concentration of 50 Wg/ml for 1 hour at 25° C. and centrifugation for 15 minutes at 3000×g. Prior to the screening ELISA, the cleared lysates were blocked by the addition of an equal volume of DMEM+10% FCS for 1 hour. For the screening ELISA, HT1080 cells were harvested with 0.05% EDTA, fixed with paraformaldehyde, diluted to 1×1O7 cells/ml in PBS and immobilized onto wells of a 96 well UV cross-link plate (Corning Costar). The wells of the UV cross-link plate were blocked with MPBS and the scFv containing blocked cleared lysates added for 1.5 hours at 25° C. The plates were washed 2× with PBS+0.1% Tween-20 and 1× with PBS, incubated with HRP conjugated a-E-tag (#27-9413-01, Pharmacia Biotech; diluted 1:5000 in MPBS with 0.1% Tween-20) for 1 hour, washed 3× with PBS+0.1% Tween-20 and 3× with PBS, developed with POD (#1 484 281, Roche) and signals read at 370 nm.

Positive clones were retested against HT1080 cells and control human fibroblasts Hs-27 (ATCC CRL-1634) using the ELISA screening procedure described above and preserved in glycerol stocks. In a typical screen, 2760 (30×92) clones were screened for binding to HT1080 cells with 5% positives defined as clones giving a background subtracted signal >0.1. 155 positive clones were retested for specific binding to HT1080 cells compared to the Hs-27 control cells with 28% positives defined as clones giving a background subtracted signal on HT1080 of at least twice the value of the signal on Hs-27 control cells.

### Example 4

### Sequencing and Large Scale Expression of Identified scFv

Sequencing of scFv1-scFv4 and their genes was performed by Sequiserve GmbH, Vaterstetten, Germany using the primer pXP2 Seq2 (5′-CCCCACGCGGTTCCAGC-3′ (SEQ ID No.: 53) and pXP2 Seq1 (5′TACCTATTGCCTACGGC-3′ (SEQ ID No.: 54). The amino acid sequence and nucleotide sequence are shown in the Figures.

Unique clones identified by sequencing were streaked out from glycerol stocks onto LB/Amp (100 ug/ml)/1% Glucose Agar plates and incubated o/n at 30° C. 10 ml LB/Amp/Glu (1%) media were inoculated with a single colony and grown o/n at 30° C. and 200 rpm shaking. The next morning the overnight cultures were placed on ice until inoculation of 1L 2×TY media supplemented with 100 Oug/ml Ampicillin and 0.1% Glucose in 2L Erlenmeyer-flasks. The cultures were grown at 25° C. shaking until an OD600 0.5-0.6 was reached and then induced with IPTG 0.1 mM final concentration. Fresh ampicillin was added to 50 g/ml and incubation was proceeded at 22° C. o/n shaking. In the morning the cultures were centrifuged at 5000×g for 15 minutes at 4° C., supernatants discarded and the pellets re-suspended carefully on ice with a pipette in 10 ml pre-cooled PBS-0.5 M Na buffer containing protease inhibitors complete (#1697498, Roche). After re-suspension was completed, bacterial suspensions were transferred to 20 ml oakridge centrifuge tubes and hen-egg lysozyme (#L-6876, Sigma) added to a final concentration of 50 tg/lnl for 1 hour on ice. The lysed bacteria were centrifuged at 20000×g for 15 minutes at 4° C. and the supernatants (lysate) transferred to a 15 ml plastic tube. For affinity purification the lysates were loaded with 1 ml/min onto 1 ml StrepTactin (# 2-1505-010, IBA) columns equilibrated with 10 column volumes (CV) PBS-0.5 M Na buffer via a parallel protein purification system. After a 10 CV wash with PBS the elution was done with 5 CV PBS/5 mM Desthiobiotin (#D-1411, Sigma) and 1 ml fractions collected. The fractions were measured at UV280, protein containing fractions were pooled and concentrated with Amicon Ultra Centrifugal Filter Devices 10.000 MWCO (#UFC801024, Millipore) at 4700×g.

The concentrated scFv were checked on 12% Bis-Tris SDS-PAGE gels stained with Coomassie Blue for purity and frozen in aliquots with 20% glycerol at −80° C.

### Example 4.1

### Reformatting of Murine scFv into Chimeric IgG (Human-Murine) and Expression

The murine scFvs consist of the sequence of a variable light and heavy chain linked by a linker sequence. The variable light chain and the variable heavy chain were amplified by PCR separately with the usage of primer, which contain restriction sites. Those restriction sites are also present in the vectors, which contain the appropriate murine constant domains for the heavy and light chain. The amplified variable domains were cut with the restriction enzymes and cloned into the cut vectors. The correct sequence was confirmed via sequencing Four vectors were used, one contained the murine constant domain of the heavy chain for IgG1 format. The second contained the murine constant domain of the heavy chain for IgG4 format and two contained the murine constant domains of lambda and kappa light chains, respectively. Different restriction sites enabled to cut the vectors and to ligate the variable domains in the vectors.

For expression of the chimeric IgGs in mammalian cell lines the vectors contained an Epstein Barr virus origin of replication (oriP sequence), which enhances the level of transcription in 293-EBNA-HEK cells, because the EBNA protein leads to the replication of the episomal vector.

A co-transfection was carried out with the vector for the heavy chain and the vector for the light chain leading to the expression of both chains in the cell and the assembly of the IgG in the Endoplasmatic Reticulum. The assembled IgG was then secreted to the medium. As transfection method Calcium-phosphate transfection was used, where a precipitate of Calcium-phosphate and the DNA is formed and incorporated into the cell. After the transfection the medium was changed to serum-free medium. Three harvests per IgG were done every 3 days. The supernatant (media) was sterile-filtrated and stored at 4° C.

For the purification of the IgGs the supernatants were purified via Protein A Sepharose either by gravity flow or by HPLC depending on the volume. For up to 200 ml a gravity flow method was used. For both purification types the supernatant was loaded on the Protein A column, washed with 50 mM Tris pH 7 buffer and eluted with 0.1 M Citrate pH-2-3. To the elution fraction 0.25 M Tris pH 9 was added leading to a pH of 5.5-6.0. Depending on the further use of the IgGs they were dialysed against PBS buffer and stored at −80° C. or 4° C.

### Example 5

### FACS Analysis for Tumor Cell Specific Binding

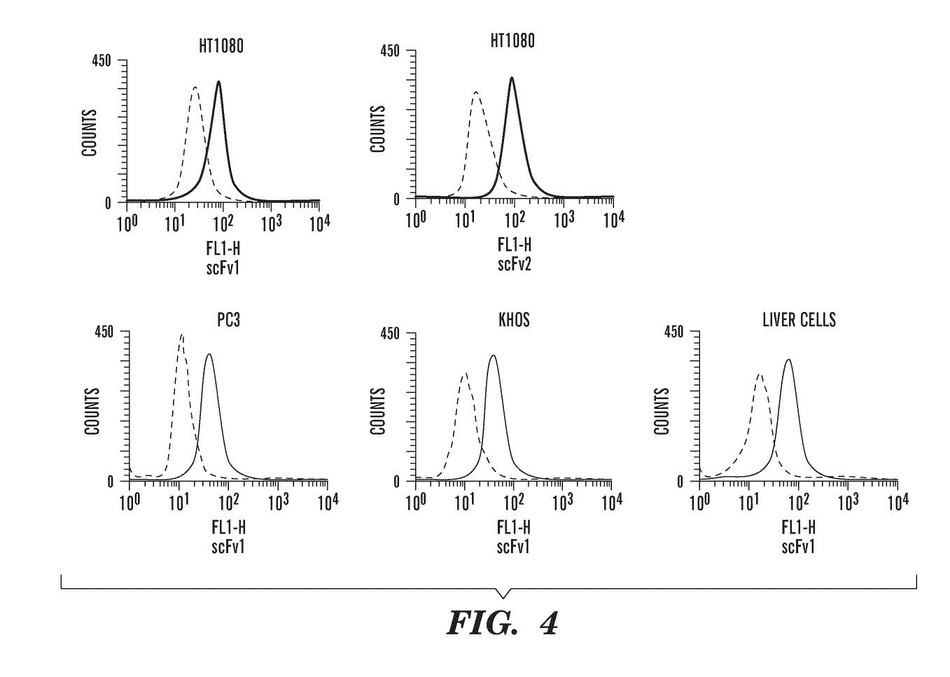

To test the ability of purified anti HT1080 scFv to bind specifically to target cells, we performed a fluorescence-activated cell sorter (FACS) analysis using HT1080 cells (ATCC CCL-121) and Hs-27 cells (106 cells/ml) as control cell line (see FIG. 4). ScFv were also tested for binding to KHOS cells (ATCC CRL-1544), PC-3 cells (ATCC CRL-1435), and chang liver cells (DSMZ ACC-57 contain impurities of Hela cells) (for all 106 cells/ml) compared to HT1080 cells. Cells were incubated with 10 llg/ml of pure scFv in CellWash (BD (Becton, Dickinson and Company) #349524) for 20 min at 4° C., washed, and bound scFv's were detected with a secondary FITC labeled anti E-tag mab (Amersham #27-9412-01). Samples were washed and analyzed on a Becton Dickinson FACSscan. FIG. 4 (upper panel) shows the log fluorescence intensity (FL1-H; x-axis) versus the relative cell numbers (counts; y-axis) for cells reacting with scFv1 and scFv2. The thin line represents the control cell line (HS-27) and the bold line the HT1080 cells. scFv1 and scFv2 specifically stain the tumor cell lines with up to 10 fold higher signals compared to the control cell line. The lower panel of FIG. 4 shows the binding of scFv1 to PC3, KHOS and chang liver cells compared to HT1080 cells. scFv1 binds to all three cells lines, but to a lower degree compared to HT1080 cells (PC3, KHOS or chang liver cells dotted line).

### Example 6

### Competition Analysis by FACS

To test the ability of the purified anti-PVR-scFv to block common PVR epitopes on the target cells, single cell suspensions of HT1080 are harvested with 0.5 mM EDTA/PBS. Approximately 1×106 cells are incubated in CellWash (BD, #349524) with 10 μg/ml scFv for one hour at 4° C. After washing with Cell Wash 1011 g/ml FITC labeled scFv is added and incubated for 20 min at 4° C. Signals of bound FITC labeled scFvs with and without pre-incubation of other PVR binders are analyzed on a Becton Dickinson FACSscan.

### Example 7

### Labeling of Antibody Fragments with FITC

scFv were labeled with fluorescein isothiocyante (FITC) (Molecular Probes, Eugene, USA #fol906) by the following method: Aliquots of a 10 mg/ml solution of FITC in dimethyl sulfoxide were added to 1001 lg of scFv dissolved in PBS/0.5M NaHCO3, pH 9.5 in a ratio of 30:1 (FITC: scFv1). The sample was incubated for two hours at room temperature with agitation, free FITC was separated using desalting columns (2 Micro Spin G-25, Pharmacia 27-5325-01). The ratio of labeling was determined via mass spectrometry and via UV/VIS spectroscopy, whereby the protein concentration was calculated at 280 nm and the FITC concentration at 494 nm.

### Example 8

### Invasion Assay for Identification of Inhibitory Molecules

The ChemoTxs system (Neuro Probe Inc. #106-8, Gaithersburg) may be used as a disposable chemotaxis/cell migration chamber in a 96 well format with an 8 um filter Track etched Polycarbonate pore size, 5.7 mm diameter/site.

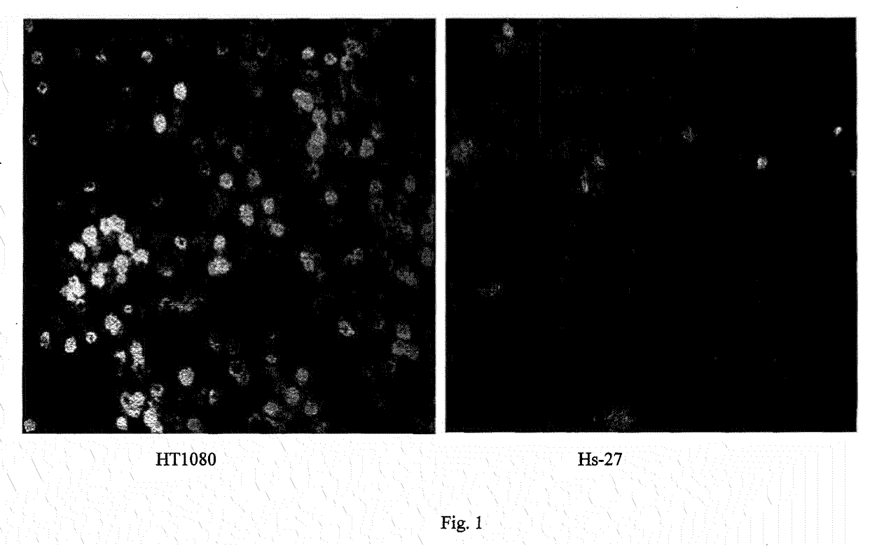

13, 3 pul of 0.3 mg/ml Matrigel (Matrigel is a solubilized basement membrane preparation extracted from the Engelbreth-Holm-Swarm (EHS) mouse sarcoma, a tumor rich in extracellular matrix proteins. Its major component is laminin, followed by collagen IV, heparan sulfate proteoglycan, entactin and nidogen. It also contains TGF-alpha fibroblast growth factor, tissue plasminogen activator, and other growth factors which occur naturally in the EHS tumor) (Becton Dickenson, BD #356234) diluted in Dulbeccos PBS (Gibco #14040-091) is applied on the membrane filter of the 96-well plate on row B-H and on row A 1, 2, ug/site of collagen S type I (Roche #10982929) is diluted in 0.05 M HCl (Sigma #945-50) and is incubated over night at 20° C. in a desiccator for gelation. HT1080 cells are grown to 70-80% confluence in DMEM supplemented with GlutamaxI (862 mg/l (Gibco #31966-021) with 10% FCS (Gibco #10270106). The cells are washed 2× with DMEM/GlutamaxI/0. 1% BSA (Sigma #A-7030) then labeled in situ with Bisbenzimide H 33342 (Sigma #B-2261) are diluted 1:100 in DMEM/GlutamaxI/0. 1% BSA for 15 min at 37° C., 7.5% C02. Cells are washed 2× with DMEM/GlutamaxI/0. 1% BSA and are loaded with DMEM/GlutamaxI/0.1% BSA for 15 min at 37° C., 7.5% COx for recovering. After washing 2× with PBS w/o Ca2+, Mg2+ (Gibco, 10010-015), the cells are detached with 0.5 mM EDTA (Sigma #E8008), are collected with Dulbeccos PBS/0.1% BSA/10 mM Hepes (Gibco #15630-056), are washed 2× with Dulbeccos PBS/0.1% BSA/10 mM Hepes, are suspended in Dulbeccos PBS/0.1% BSA/10 mM Hepes and are diluted to 6.7×106 cells/ml with Dulbeccos PBS/0.1% BSA/10 mM Hepes. 6.7×106 cells/ml are incubated 1:1 with 4011 gel of a control scFv as a negative control for inhibition of invasion and with HT1080 specific scFv for 1 h on ice. After dilution to 6.7×105 cells/ml with DMEM/GlutamaxI/0.1% BSA, HT1080 cells and HT1080 cell/scFv dilutions are pipetted in triplicate onto the chemotaxis chamber (row B-H) at a density of 3.4×104 cells/well and are incubated for 6 h at 37° C., 7.5% CO2. DMEM/GlutamaxI with 5% FCS is used as a chemo attractant in the lower chamber. A standard curve from 1×104 to 4×104 cells/site is performed on collagen S type I coated row A of the chemotaxis chamber. DMEM/GlutamaxI/0.1% BSA is used in the lower chamber (cells are not migrating). After scraping the non-migrating cells from the top of the membrane (except the Standard curve on row A) fluorescence of cells, which had migrated through the membrane (not migrated in case of the Standard curve), is measured on the Fluostar Galaxy (bMG) microplate reader using excitation/emission wavelengths of 370/460 nm. (FIG. 1).

### Example 9

### Invasion Assay for Target Identification with CALI

This Example is in principle identical to Example 8, except for the use of FITC-labeled scFv and the additional integration of the CALI process within the invasion assay. In the context of the CALI process the scFv was labeled with a CALI inducible label.

The ChemoTxe system (Neuro Probe Inc. #106-8, Gaithersburg) may be used as a disposable chemotaxis/cell migration chamber in a 96 well format with an 8, um filter Track etched Polycarbonate pore size, 5.7 mm diameter/site.

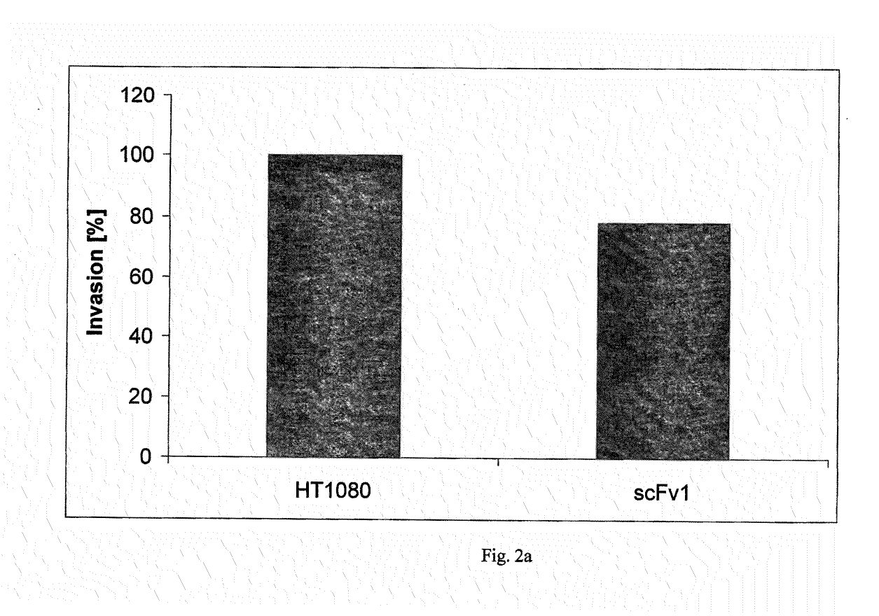

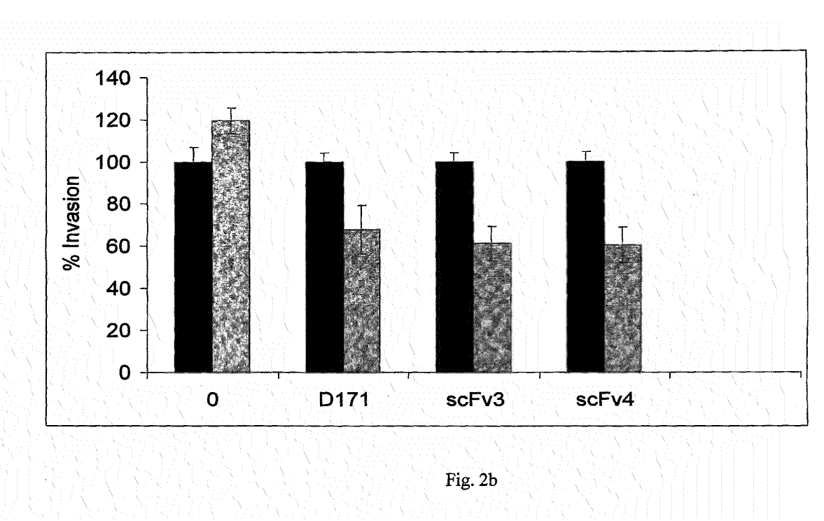

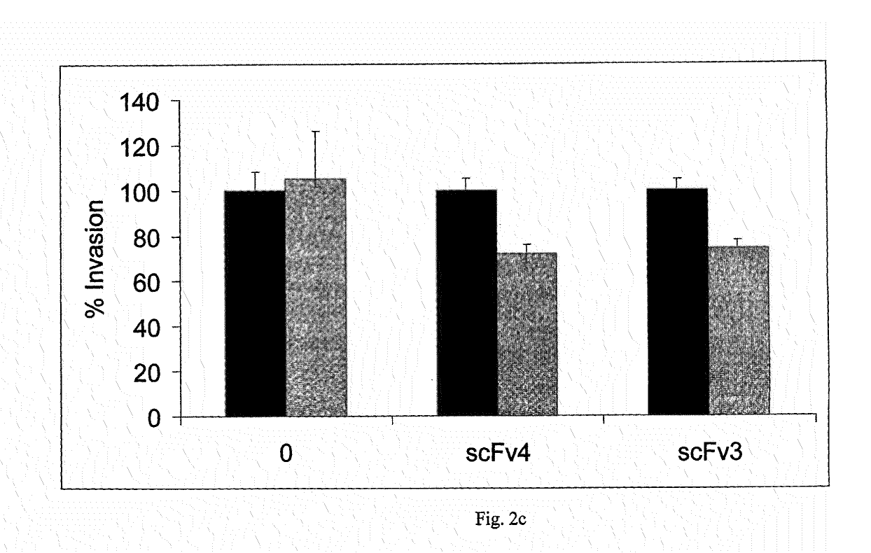

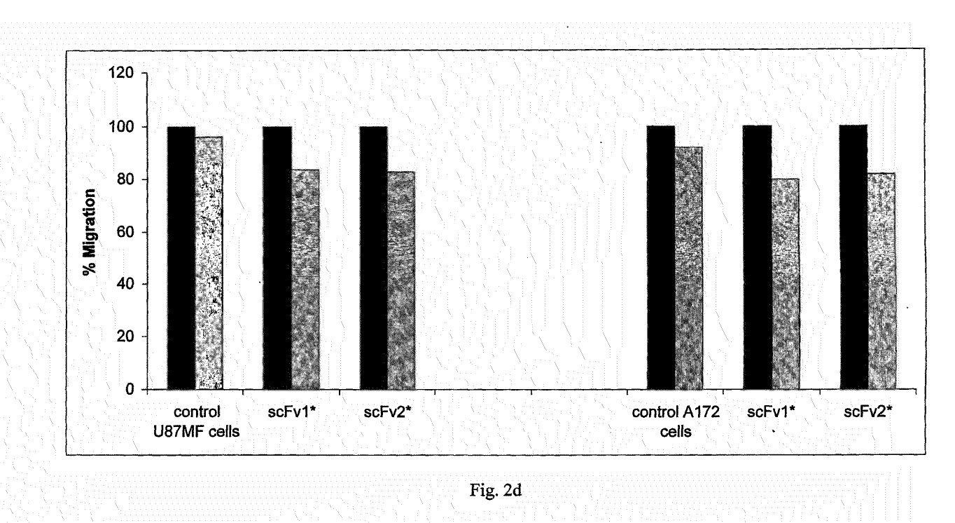

13.3 μl of 0.3 mg/ml Matrigel (see Example 8) diluted in Dulbeccos PBS (Gibco #14040-091) is applied on the membrane filter of the 96-well plate on row B-H and on row A 1.2 Fg/site of collagen S type I (Roche #10982929) is diluted in 0.05 M HCl (Sigma #945-50) and is incubated over night at 20° C. in a desiccator for gelation. HT1080 cells were grown to 70-80% confluence in DMEM supplemented with GlutamaxI (862 mg/l (Gibco #31966-021) with 10% FCS (Gibco #10270106). The cells were washed 2× with DMEM/GlutamaxI/0. 1% BSA (Sigma #A-7030) then labeled in situ with Bisbenzimide H 33342 (Sigma #B-2261) and diluted 1:100 in DMEM/GlutamaxI/0. 1% BSA for 15 min at 37° C., 7.5% CO2. Cells were washed 2× with DMEM/GlutamaxI/O. 1% BSA and loaded with DMEM/GlutamaxI/0. 1% BSA for 15 min at 37° C., 7.5% C02 for recovering. After washing 2× with PBS w/o Ca2+, Mg2+ (Gibco, 10010-015), the cells were detached with 0.5 mM EDTA (Sigma #E8008), collected with Dulbeccos PBS/0.1% BSA/10 mM Hepes (Gibco #15630-056), washed 2× with Dulbeccos PBS/0.1% BSA/10 mM Hepes, suspended in Dulbeccos PBS/0.1% BSA/10 mM Hepes and diluted to 6.7×106 cells/ml with Dulbeccos PBS/0.1% BSA/l OmM Hepes. 6.7×106 cells/ml are incubated 1:1 with 40 llg/ml of FITC-labelled anti-beta1 integrin monoclonal antibody (JB1, Chemicon #MAB1963) as a control for inhibition of invasion after CALI and with HT1080 specific FITC labelled scFv for 1 h on ice. 1.3×105 HT1080 cells/well or HT1080 cell/scFv or Ab dilution were pipetted in triplicate in two 96-well plate, black, ultra thin clear flat bottom special optics (Costar #3615). One plate was kept on ice in the dark while the other plate was irradiated on an ice block with continuous wave laser at 488 nm (0.5 W, 30 sec). After dilution to 6.7×105 cells/ml with DMEM/GlutamaxI/O. 1% BSA, HT1080 cells and HT1080 cell/scFv dilutions were pipetted in triplicate (non irradiated triplicate beside irradiated triplicate) onto the chemotaxis chamber (row B-H) at a density of 3.4×104 cells/well and incubated for 6 h at 37° C., 7.5% CO2. DMEM/GlutamaxI with 5% FCS is used as a chemo attractant in the lower chamber. A standard curve from 1×104 to 4×104 cells/site was performed on collagen S type I coated row A of the chemotaxis chamber. DMEM/GlutamaxI/O. 1% BSA was used in the lower chamber (cells are not migrating). After scraping the non-migrating cells from the top of the membrane (except the Standard curve on row A) fluorescence of cells, which had migrated through the membrane (not migrated in case of the Standard curve), was measured on the Fluostar Galaxy (bMG) microplate reader using excitation/emission wavelengths of 370/460 nm. The result of the invasion assay with laser irradiation is shown in FIG. 2a. scFv1 inhibits the invasion by about 22%.

### Example 9.1

### Invasion Assay for Target Identification with CALI (with Blue-Filtered Light)

This Example is in principle identical to Example 9, except for the irradiation of samples with blue-filtered 300 W light instead of irradiation with a laser.

HT-1080 cells were incubated with 20_g/mL of fluorescein isothiocyanate (FITC)-labeled antibody for 1 hour, then irradiated with blue filtered 300 Watt light for another hour. The cells were then assayed for invasiveness through a Matrigel coated porous membrane (Neuro Probe Inc.) for 6 hours. Bars represent percent invasion normalized to no irradiation conditions standard error, and represent at least two independent experiments in triplicate. The left bar in FIG. 2b (0) shows the invasion of cells that were not treated with any antibody, D171 (NeoMarker # MS-465) is a commercially available monoclonal antibody directed against PVR/CD155. Both scFv, scFv3 and scFv4, inhibited the invasion of HT1080 cells by about 30%. See FIG. 2b.

### Example 9.2

### Invasion Assay with Breast Cancer Cells

This Example is in principle identical to Example 9.1, except for the use of MDA-MD231 breast cancer cells instead of HT1080 cells. scFv3 and scFv4 showed an inhibition of invasion of MDA-MD231 cells by about 23%. See FIG. 2c.

### Example 9.3

### Migration Assay with Glioblastoma Cells (U87MG and A172 Cells)

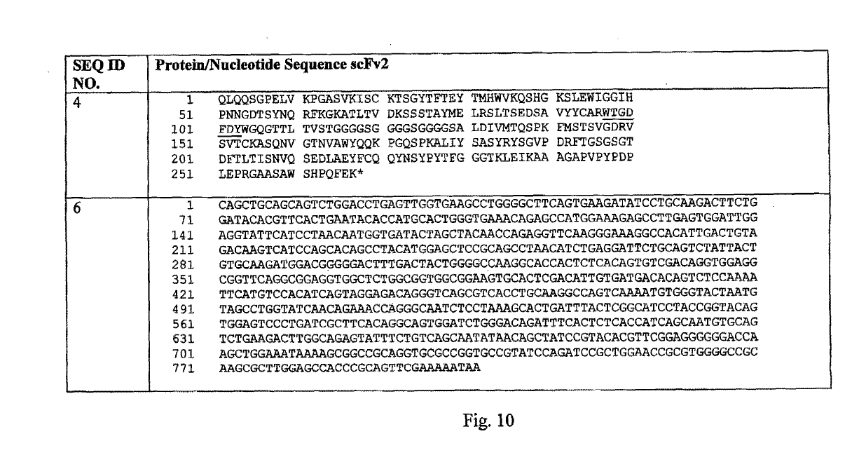

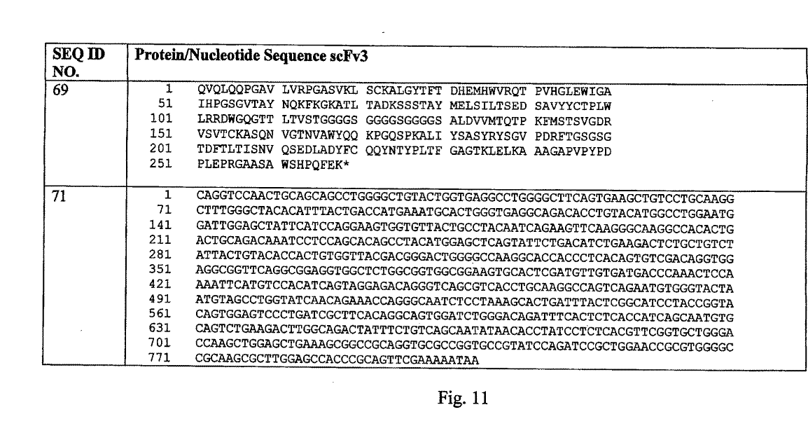

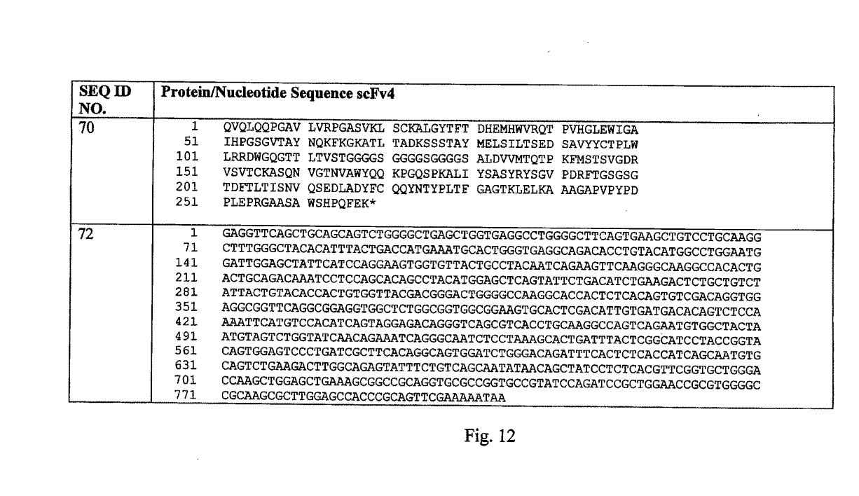

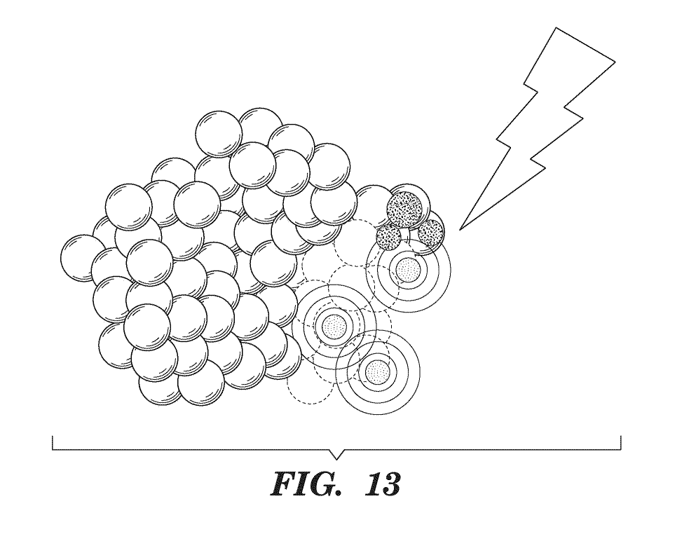

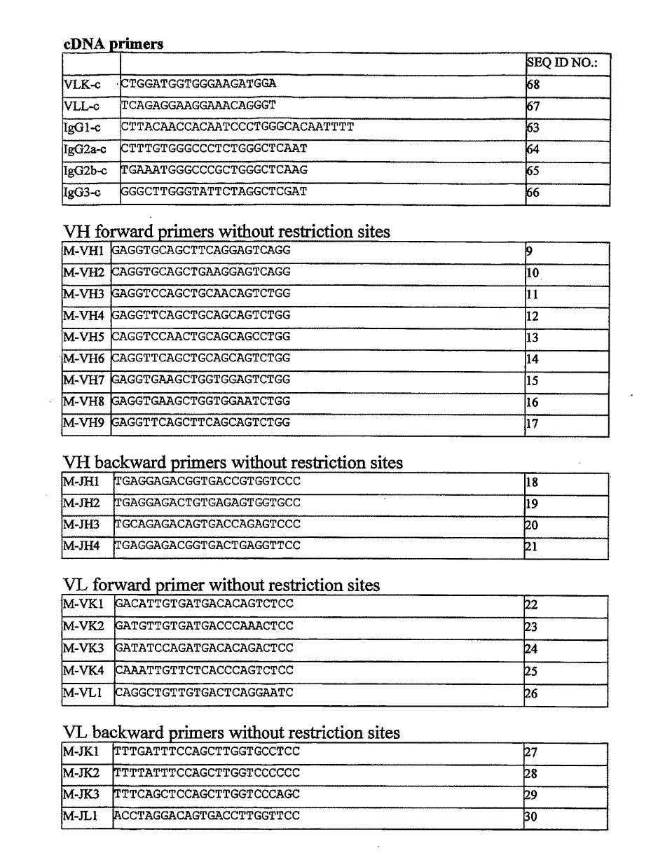

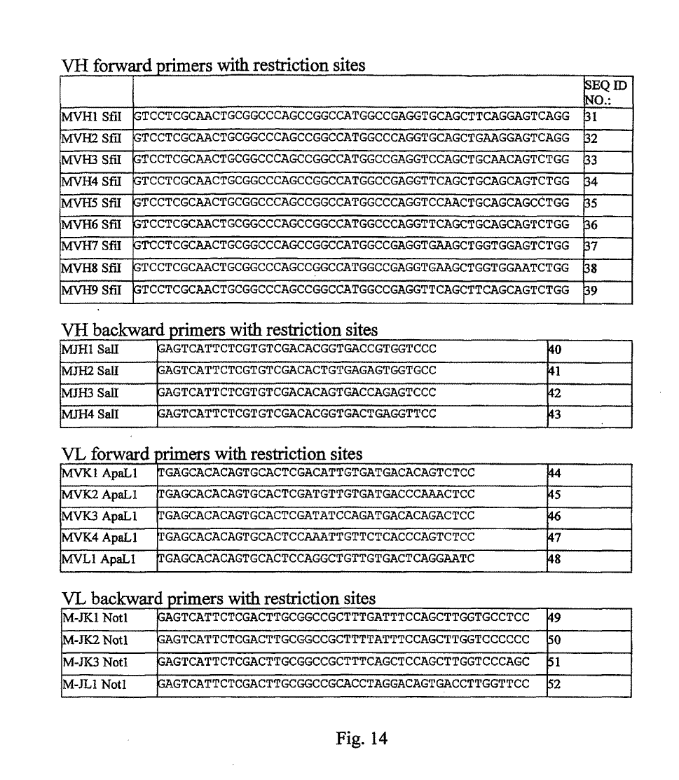

This Example is in principle identical to Example 9.1, except for the use of Beckton Dicldnson plates instead of the ChemoTxs system. These Beckton Dickinson plates comprise inserts, which consist of PET membranes with a blue dye (Fluoroblok), which allow to measure fluorescence from top or bottom only. 25,000 cells (U87MG and A172) were used in each well. scFv1 showed an inhibition of migration of U87MG cells by about 15% (data not shown). scFv1* and scFv2* showed an inhibition 16% and 17% on U87MG cells and 20% and 18% on A172 cells (the symbol“*” indicates that the respective scFv—as identified by its number as shown in FIG. 10 and FIG. 11—was cloned into an IgG format, see Example 4.1) Results are shown in FIG. 2d.

It was further investigated how PVR expression varies between different cells lines and correlates with migration behavior. PVR expression levels were assayed by immunoblot across four glioblastoma (GBM) cell lines, which were then assayed for migration behaviour in a migration assay. Expression of PVR was highest in HT1080 cells, moderate in U87 and U251 GBM cells, and weakest in SNB19 and A172 GBM cells. This expression level did not correlate with migration behavior among the GBM cell lines as U87 (moderate) and A172 (weak) invaded much more rapidly than SNB19 (weak) or U251 (moderate). (Data not shown). Of note, HT1080 cells are immortalized through N-ras overexpression and both U87 and U251 GBM cells have been reported to have high constitutive levels of ras activity. Since these observations correlate with PVR expression levels, it is tempting to speculate that ras may somehow play a role in regulating PVR expression.

### Example 10

### MTS Viability Assay

Viable cells were detected by measuring the conversion of the tetrazolium dye MTS (MTS, Celltiter Aqueous one, Promega #G4000) to formazan. HT1080 cells and HT1080 cell/scFv dilutions (obtained from the dilutions prepared in the Adhesion assay) were pipetted in triplicate at a density of 3.4×104 cells/well and were plated in a 96-well plate (black, ultra thin clear flat bottom, special optics, Costar #3615). 10 u. 1 MTS was added to each well and incubated for 1 hour at 37° C., 7.5% CO2. Absorbance was measured at 492 nm with the Fluostar Galaxy (bMG) microplate reader. For the tested scFv1 and scFv2, no effect on viability of cells was seen.

### Example 11

### Cell-Matrix Adhesion Assay for Identification of Inhibitory Antibody Fragments

21 wells of a 96-well flat bottom plate (Costar #3614) are coated with one matrix protein selected from collagen S type I 1 llg/well (Roche #10982929), collagen type IV 1 llg/well (Rockland 009-001-106), fibronectin lu. g/well (Sigma F2518) and laminin 1, ug/well (Roche 1243217) in Dulbeccos PBS (Gibco #14040-091), respectively, at 4° C. over night. At the same time 3 wells in row A are coated with 2% BSA (Sigma #A-7030)/Dulbeccos PBS for a blank value. Wells are washed twice with Dulbeccos PBS, and blocked with 2% BSA (Sigma #A-7030)/Dulbeccos PBS for 1 h at 37° C. and washed with Dulbeccos PBS. HT1080 are harvested, stained with 2.5 mM (final concentration) Calcein AM (Molecular Probes C-3099), washed twice with PBS w/o CaCl2 w/o MgC12 (Gibco 10010-015) and diluted to 1.5×105/ml in buffer (0.5% BSA (Sigma #A-7030)+1 OmM Hepes+DMEM (Gibco 31966-02)). HT1080 cells are mixed with 10, g/ml scFv and incubated for 30 min on ice. The HT1080 cells alone and HT1080/scFv dilutions are pipetted in triplicate at a density of 1.5×104 cells/well and incubated for one hour at 37° C., 7.5% C02. After two additional washing steps with Dulbeccos PBS, where non-adherent cells are washed away, a Standard curve from 3.7×103 to 1,5×104 stained cells/well diluted in Dulbeccos PBS may be performed in triplicate in row A. Washed wells are filled with 100p1 Dulbeccos PBS and the absorbance of attached cells and of the Standard curve is measured on the Fluostar Galaxy (bMG) microplate reader using excitation/emission wavelengths of 485/520 nm.

### Example 12

### Cell-Matrix Adhesion Assay for Target Identification with CALI

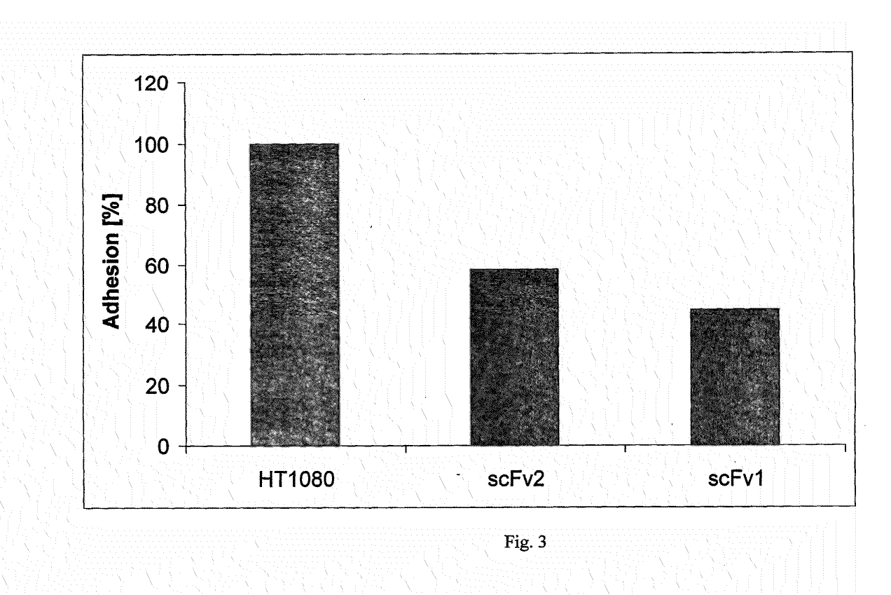

96-well plates (TPP #9296) (cell culture treated) were coated in Row B-H with collagen S type I 1 ug/well (Roche #10982929) in Dulbeccos PBS (Gibco #14040-091) and in Row A well 10-12 were coated with 2% BSA (Sigma #A-7030)/Dulbeccos PBS at 4° C. over night. The plate was washed with Dulbeccos PBS, blocked Row B-H and Row A well 10-12 with 2% BSA/Dulbeccos PBS for 1 h at 37° C. and washed again with Dulbeccos PBS. HT1080 cells were grown to 70-80% confluence in DMEM supplemented with GlutamaxI (862 mg/l (Gibco #31966-021) with 10% FCS (Gibco #10270106). The cells were washed 2× with DMEM/GlutamaxI/0.1% BSA (Sigma #A-7030) then labeled in situ with Bisbenzimide H 33342 (Sigma #B-2261) were diluted 1:100 in DMEM/GlutamaxI/0. 1% BSA for 15 min at 37° C., 7.5% C02. Cells were washed 2× with DMEM/GlutamaxI/0. 1% BSA and loaded with DMEM/GlutamaxI/0. 1% BSA for 15 min at 37° C., 7,5% C02 for recovering. After washing 2× with PBS w/o Ca2+, Mg2+ (Gibco, 10010-015), the cells were detached with 0.5 mM EDTA (Sigma #E8008), collected with Dulbeccos PBS/0.1% BSA/10 mM Hepes (Gibco #15630-056), washed 2× with Dulbeccos PBS/0.1% BSA/10 mM Hepes, suspended in Dulbeccos PBS/0.1% BSA/10 mM Hepes and diluted to 6.7×106 cells/ml with Dulbeccos PBS/0.1% BSA/10 mM Hepes. 6.7×106 cells/ml were incubated 1:1 with 40, ug/ml of FITC-labeled anti-beta1 integrin monoclonal antibody (JB1, Chemicon #MAB1963) as a control for inhibition of adhesion after CALI and with HT1080 specific FITC labeled scFv for 1 h on ice. 1.3×105 HT1080 cells/well or HT1080 cell/scFv or Ab dilution were pipetted in triplicate in two 96-well plate, black, ultra thin clear flat bottom special optics (Costar #3615). One plate was kept on ice in the dark while the other plate was irradiated on an ice block with continuous wave laser at 488 nm (0.5 W, 30 sec). After dilution to 6.7×105 cells/ml with DMEM/GlutamaxI/0. 1% BSA, HT1080 cells and HT1080 cell/scFv dilutions were pipetted in triplicate (non irradiated triplicate beside irradiated triplicate) onto the coated and blocked plate. In Row A well 10-12 6.7×105 cells/ml with DMEM/GlutamaxI/0.1% BSA were pipetted as a background control. Plate was incubated for 1 h at 37° C., 7.5% C02 and washed 2× with Dulbeccos PBS, where non-adherent cells were washed away. In Row A well 1-9 a standard curve from 1×104 to 4×104 cells/well was performed, in all other wells 50 1 ll Dulbeccos PBS was pipetted. Fluorescence of cells, which had adhered to the Collagen S type 1 (not adhered in case of the Standard curve), was measured on the Fluostar Galaxy (bMG) microplate reader using excitation/emission wavelengths of 370/460 nm. scFv1 inhibited the adhesion by 55.5% and scFv2 inhibited the adhesion by 42%. Results for both scFv's are shown in FIG. 3.

### Example 13

### Immunoprecipitation

HT1080 and Hs-27 cells (108) were lysed in 3 ml 50 mM Tris-HCl, pH 8.0, 150 mM NaCl, 1% Triton X-100 (v/v) containing protease inhibitor cocktail (1 pill in 50 ml buffer) (Boehringer Mannheim, Cat-No. 1697498) and 100 uM Pefabloclc (Roth, Cat.-No. A154.1). Lysates were pre-incubated for 2 h at 4° C. with Streptactin-coupled magnetic beads and the supernatants used for the immunoprecipitation reactions. HT1080 specific scFv (50 pg/1 mg cell extract) were added to the cleared lysates, samples rotated for 2 h at 4° C., placed on a magnet to collect the beads at the tube wall, the beads were washed 4 times with 1 ml volume of PBS+0.1% Tween buffer per wash, before the complexes were isolated by elution from the streptactin magnetic beads with 20 Ill O. 1 M Citrate pH 3.1. The eluate was immediately neutralized by adding 5 ul 1M Tris-HCl pH 8.0. The immuno-complexes were separated by SDS-PAGE and silver stained for MS analysis.

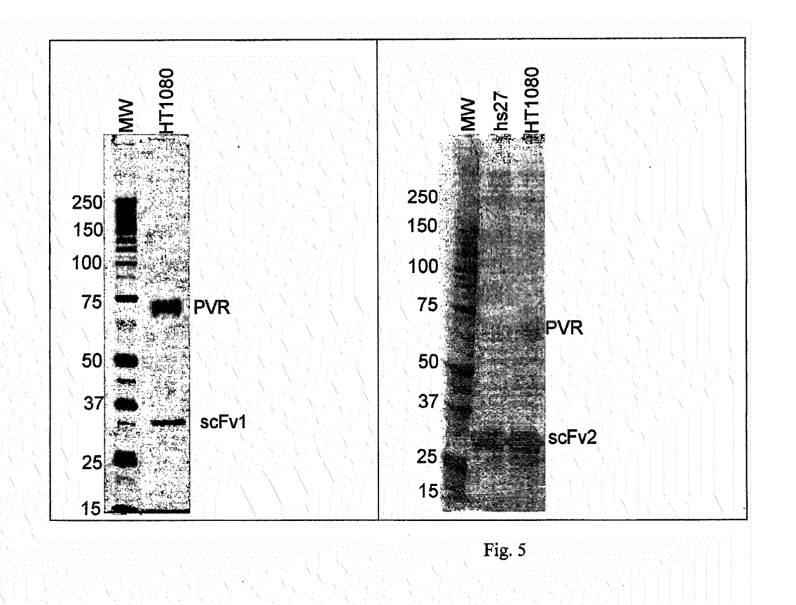

Both, scFv1 and scFv2, pulled down a protein, detected as a band on SDS-PAGE by silver staining at a molecular weight of about 70 kDa. This band was only detected in the HT1080 cell extract and not in the Hs-27 cells (control cells), see FIG. 5.

### Example 14

### Protein Identification Via Mass Spectroscopy

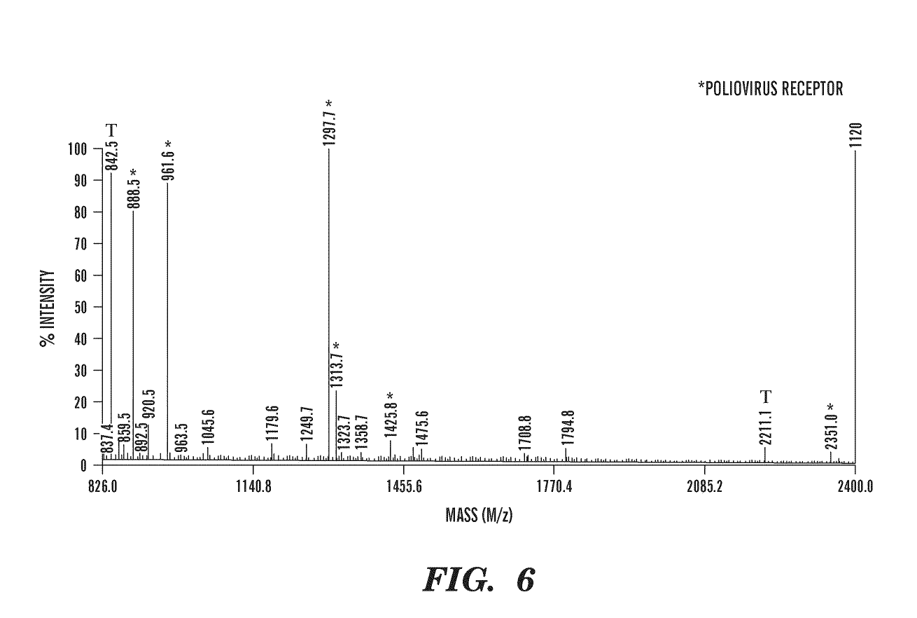

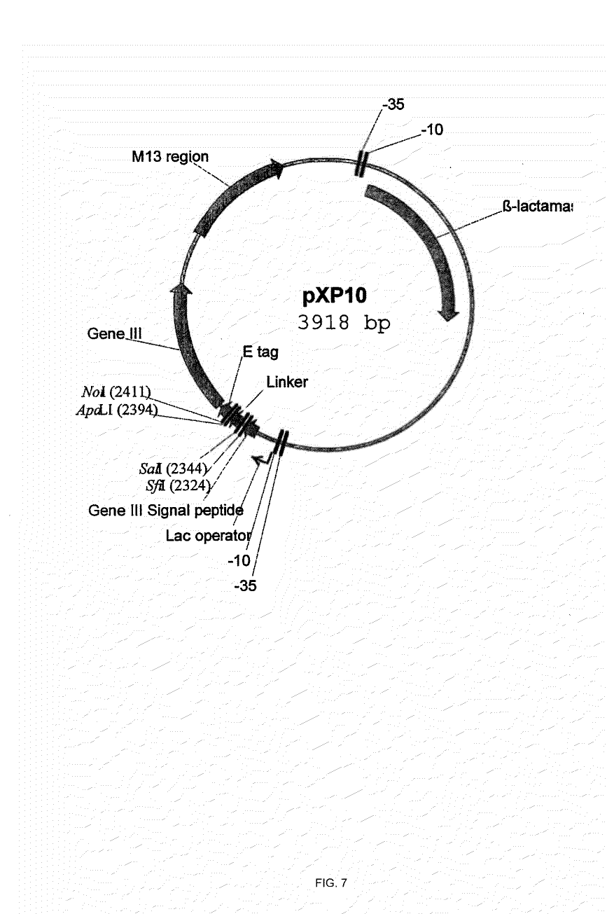

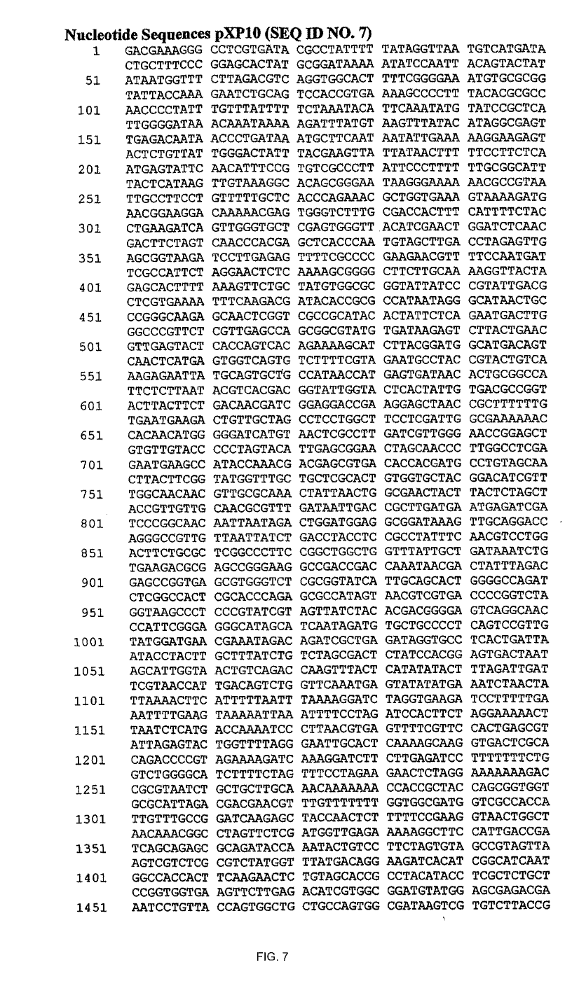

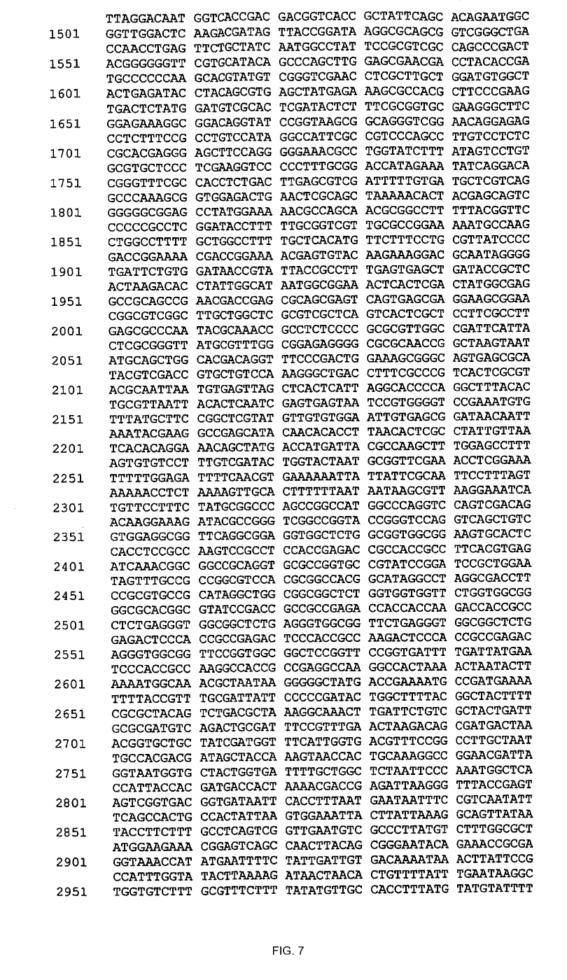

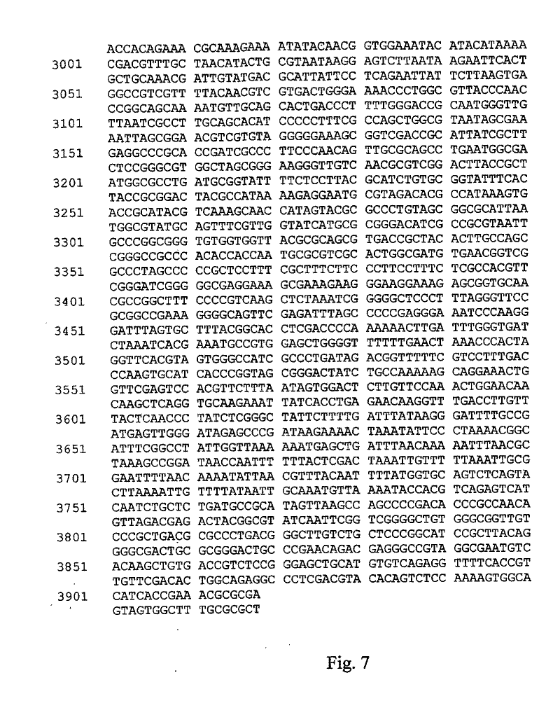

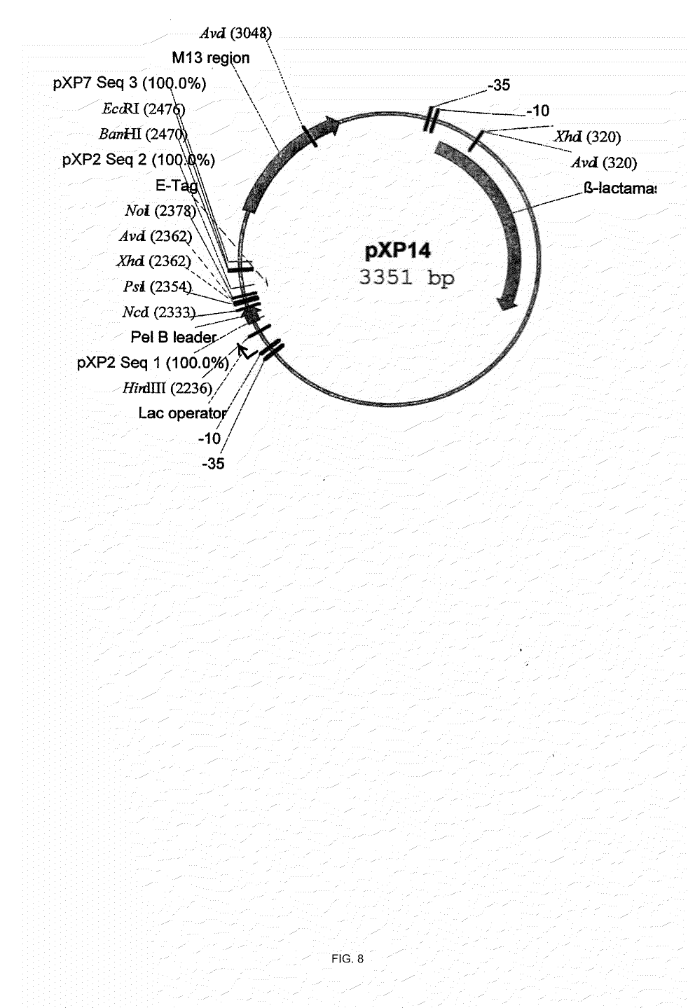

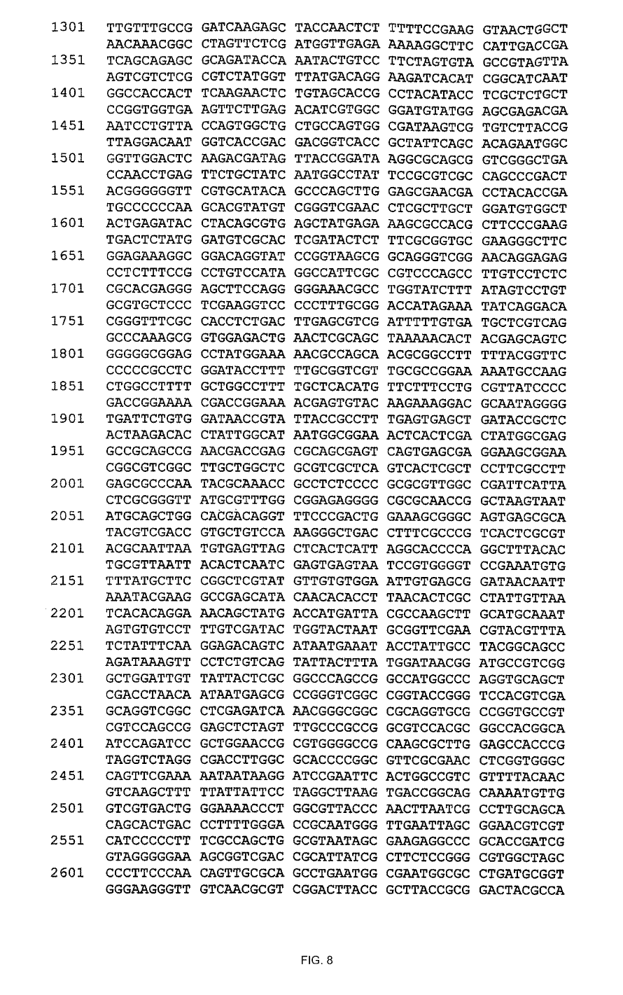

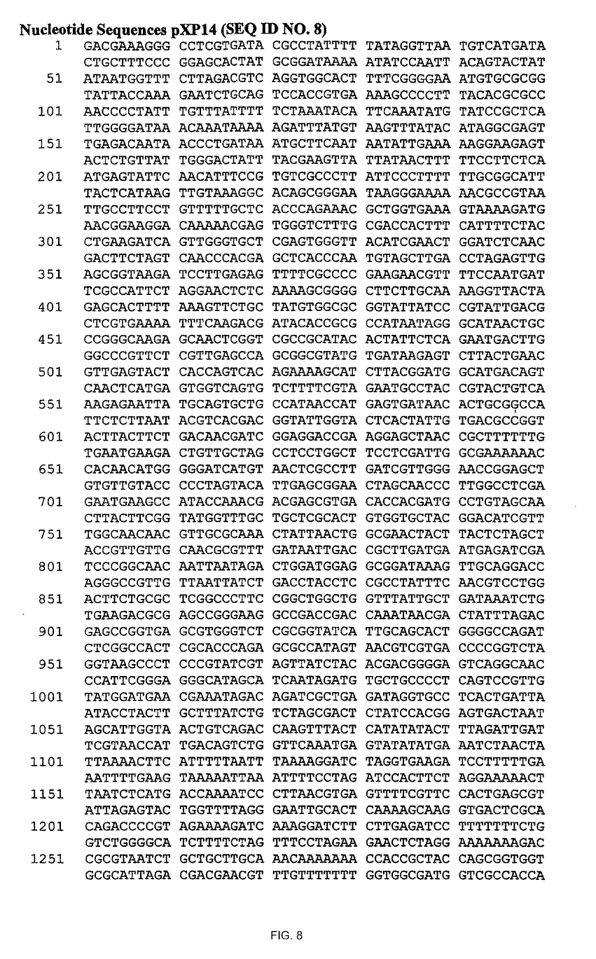

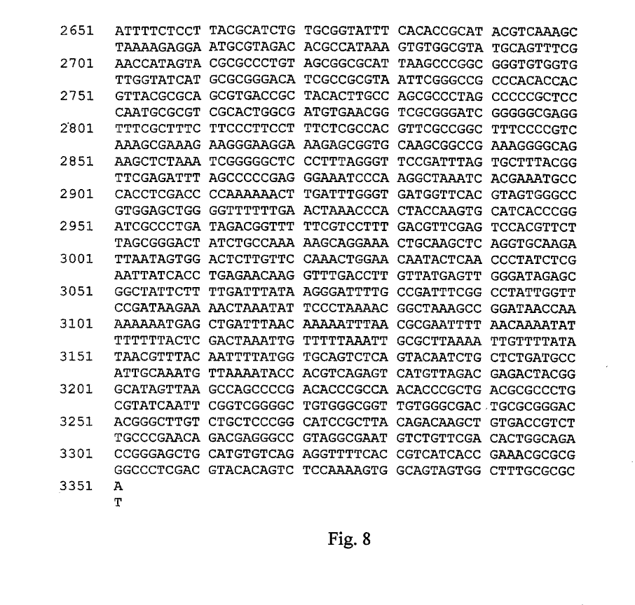

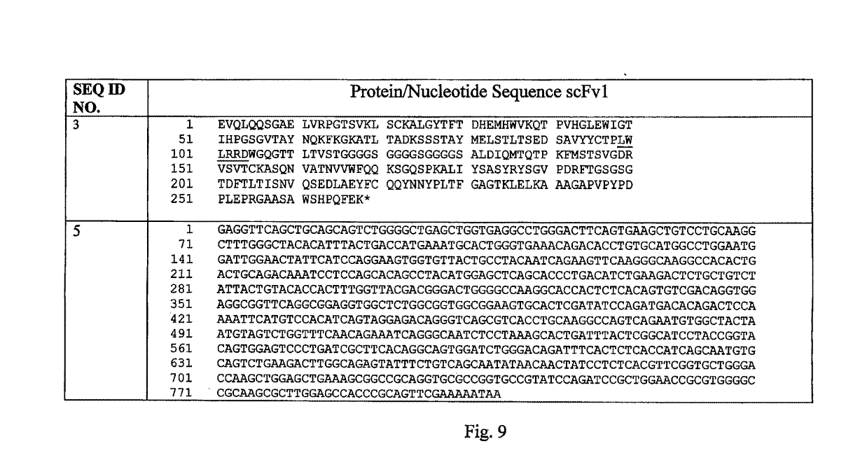

The gel bands obtained from immunoprecipitations followed by SDS PAGE were subjected to a tryptic in-gel digest over night at 37° C. Peptides were extracted using 5% formic acid and the resulting peptide mixture was desalted using ZipTip, uC18 (Millipore) and eluted first with 2 Ill of 30% ACN/0.1% TFA, then with 2 pLi of 70% ACN/0.1% TFA. The two fractions were pooled and one microliter of the obtained peptide mixtures was mixed in a 1:1 ratio with a solution of a-cyano-4-hydroxycinnamic acid (3 mg/ml), co-crystallized on a Teflon-coated stainless steel target and analyzed on a MALDI-TOF instrument yielding peptide mass fingerprints (PMF) in a mass range of m/z 800-3000. The obtained PMF were used to search all entries for the species Homo sapiens in the NCBI and SwissProt databases. In all cases, only peptides matching a given protein with a mass deviation of less than 10 ppm were considered for identification. 4 to 6 peptides were detected, which matched the PVR in the database, with the difference between measured and real mass being always below +/−1 Oppm. In some experiments these 4 peptides were observed as 2 doublets, each representing 1 peptide with or without +16 Da, due to methionine oxidation. When a de-glycosilation of the peptide mixture was carried out, an additional peptide was observed, which contains Asn120, annotated in the database as being glycosylated. A spectra is shown in FIG. 6.

Another sample was also measured by nanoES-MSMS. 4 peptides were detected with this method. Three of them were fragmented by CID (collision induced dissociation) for MSMS analysis. For each fragment a sequence tag was obtained and used for a data base search. Three peptides matched the known poliovirus receptor, one of them with an oxidized Methionine.

### Example 15

### Methods for Epitope Mapping

An epitope mapping may be carried out according to one of the following methods:

### Example 15.1

### “Classical” Epitope Mapping

Defined fragments of the cDNA for the antigen of interest are expressed as recombinant fusionproteins or proteins and probed in various assays such as Westernblot or ELISA.

### Example 15.2

### Phage Display Technology

The technique of epitope mapping using random peptide phage display libraries was developed to clone small random fragments of the cDNA for the antigen of interest into the phage protein pIII of filamentous phages and display them on the surface of the phage (Fack et al., (1997) J. Immunol. Methods 7, 43-52). Epitope-displaying phages can be captured with antibodies in a procedure called “bio-panning”. Sequencing of the inserts of the corresponding phages gives some information on the epitopes. This procedure is used to identify conformational epitopes.

### Example 15.3

### Peptide Scan Technology

It is based on the synthesis of immobilized peptides on activated membranes using the Fmoc chemistry. Amino acid solutions are applied to the activated membrane leading to a peptide bond between the amino-group on the membrane (the membrane is activated with PEG) and the activated carboxy-group of the applied amino acid. After each cycle a specific washing procedure, acetylation, deprotection and monitoring of free amino-groups is performed. In contrast to the in vivo protein-synthesis membrane bound oligo-peptide chains are stepwise synthesized from C- to the N-terminus. Oligo-peptides containing natural as well as modified amino acids can be synthesized up to a length of 20 amino acids. Following synthesis the membranes are equilibrated and unspecific binding sites are blocked. After incubation with the antibody of interest and several washing steps the detection is performed using an HRP-conjugated secondary antibody in combination with the ECL-System. Membranes can be stripped, regenerated, and re-used up to 10 times depending on the antibody. Small overlapping oligo-peptides that ideally cover the complete amino acid sequence of the antigen of interest are synthesized on a solid support. This method is used for the identification of linear epitopes on the amino acid level. It also used for rapid mutational studies.

### Example 16

### Depletion of Cellular PVR Protein by RNAi Against PVR mRNA

The following ribo-oligonucleotides can be purchased from, e.g. Proligo (Hamburg, Germany, www. proligo. com), Pierce Chemical (part of Perbio Science, Rockford, Ill., www. perbio. com), or another supplier of RNA oligonucleotides:

(SEQ ID No.: 55)

1A (5′-CCAGTCACTTGTCTGGAGCTT-3′),

(SEQ ID No.: 56)

2A (5′-GAGCTTGAAGAAGTGGGTATT-3′),

(SEQ ID No.: 57)

1B (5′-TGCTGGTGGCATTACTGGTGC-3′),

(SEQ ID No.: 58)

2B (5′-GCTGTCCTGGCCACCCCGTGGAA-3′),

(SEQ ID No.: 59)

3A (5′-GGCGCGGAGCTGCGGAATGCCT-3′),

(SEQ ID No.: 60)

4A (5′-CCTCGCTGAGGATGTTCGGGTT-3′),

(SEQ ID No.: 61)

3B (5′-CCCGTGAACACAGCTGAGGTT-3′),

(SEQ ID No.: 62)

4B (5′-CAAGGTGGACCCACGAGAGCTTT-3′),

(SEQ ID No.: 73)

5A (5′-CAACUUUAAUCUGCAACGUTT-3′),

(SEQ ID No.: 74)

5B (5′-TTGUUGAAAUUAGACGUUGCA-3′).

The oligonucleotides 1A and 2A anneal to form siRNA directed against the PVR mRNA, the oligonucleotides 1B and 2B anneal to form a negative control for the pair above, as the nucleotides underlined are mismatches to the PVR mRNA. Also the underlined oligonucleotides 3A and 4A anneal to form siRNA directed against the PVR mRNA, the oligonucleotides 3B and 4B anneal to form a negative control for the 3A/4A-pair, as the nucleotides in bold are mismatches to the PVR mRNA. SA/B targets the PVR mRNA sequence (GI 19923371) at position 1094.

The oligonucleotide pairs 1A/2A, 1B/2B, 3A/4A, 3B/4A are annealed using Protocol 2 on page 203 of Elbashir et al., Methods (2002) 26: 199-213. When working with RNA special care is used to avoid RNAse contamination of the oligonucleotides. DEPC-treated water is used for preparing buffers. Gloves are always worn to avoid RNAse-contamination by contact with the skin. For detailed instruction on RNA-related work consult, e.g. Chapter 7.3 and 7.4 of the 2 nd edition of Sambrook et al. “Molecular Cloning: A Laboratory Manual” (1989), Cold Spring Harbor Laboratory Press.

HT1080 cells are grown to about 90% confluence in DMEM supplement with Glutamax (Gibco #31966-021) with 10% FCS (Gibco #10270106). 24 h before transfection with the annealed oligonucleotide pairs described above, the 90% confluent cells out of a 175-ml cell culture flask are harvested, e.g. trypsinized with 10 ml trypsin-EDTA solution (Life Technologies), gently centrifuged and resuspended in 10 ml DMEM. The cell suspension is diluted 1:10 with fresh DMEM medium with 10% FCS without antibiotics and transferred as 500 u. 1 aliquots into each well of a 24-well plate. 24 h after seeding the cells a confluency of about 50% is reached.

Separate wells of these cells are then transfected with the annealed oligonucleotide pairs described above according to Protocol 5 on page 207 of Elbashir et al., Methods (2002) 26, 199-213.

After 2 days of incubation according to step 5 of protocol 5 mentioned above, the cells are harvested from the wells and the cells of each well are processed for Western blot, e.g. samples (corresponding to the same number of cells) from a well transfected without oligonucleotides (untreated cells), a well transfected with the oligonucleotide pair 1A/2A, the pair 1B/2B (negative control 1/2), the pair 3A/4A and the pair 3B/4B (negative control 3/4) are loaded on an acrylamide gel, after the run the proteins are blotted onto, e.g. nitrocellulose, e.g. using a semi-dry blotting apparatus, and the nitrocellulose membranes are probed with anti-PVR antibodies.

The amount of PVR protein detected in the samples 1A/2A or 3A/4A is reduced by more than 75% in comparison to the untreated cells. A significant reduction of the level of PVR protein is not detected in the negative controls 1B/2B and 3B/4B in comparison to the untreated cells.

### Example 17

### Inhibition of Adhesion and Invasion by RNAi Against PVR

HT1080 cells are grown to about 90% confluence in DMEM supplement with Glutamax (Gibco #31966-021) with 10% FCS (Gibco #10270106). 24 h before transfection with the annealed oligonucleotide pairs described above, the 90% confluent cells out of a 175-ml cell culture flask are trypsinized with 10 ml trypsin-EDTA solution (Life Technologies). The cell suspension is diluted 1:10 with fresh DMEM medium with 10% FCS without antibiotics and transferred as 500 ul aliquots into each well of a 24-well plate. 24 h after seeding the cells a confluency of about 50% is reached.

Separate wells of these cells are then transfected with the annealed oligonucleotide pairs described above according to protocol 5 on page 207 of Elbashir et al., Methods (2002) 26, 199-213.

After 2 days of incubation according to step 5 of protocol 5 mentioned above, the cells are labeled in situ with Bisbenzimide H 33342 (Sigma #B-2261) for 15 min at 37° C., 7,5% COz. After washing, the cells are dissociated with 0.5 mM EDTA/PBS (Sigma #E8008), washed and suspended in 0.1% BSA (Sigma #A7030)/DMEM. The cells are then diluted to 6,7×105 cells/ml with 0.1% BSA/DMEM, HT1080 cells and added onto the chemotaxis chamber as described in Example 8 or on a 96 well plate as described in Example 11. Invasiveness is then determined as described in Example 8 and adhesiveness is determined as in Example 11.

The adhesiveness or invasiveness measured in at least one of the samples 1A/2A or 3A/4A is reduced by more than 40% in comparison to the untreated cells. A significant reduction of invasiveness is not detected for the negative controls 1B/2B and 3B/4B in comparison to the untreated cells.

### Example 17.1

### Inhibition of Migration by RNAi Against PVR

In a similar assay, HT1080 cells were grown to about 75% confluence in 6-well plates, transfected with 200 nM 5A/B using Oligofectamine. After 48 h cells labeled with a cell tracker orange dye and harvested with EGTA/Versene. Cells were counted and adjusted to the desired concentration for carrying out a migration assay according to Example 9.3.

### Example 18

### Immunohistochemistry Protocol for Paraffin Slides

IHC was carried out at LifeSpan BioSciences, Inc., Seattle, USA A microtome was used to cut tissue sections at 4-5 microns. The tissue sections were floated on a water bath and picked up onto slides. Slides containing paraffin sections were deparaffinized through xylene and alcohol, rehydrated, and then subjected to the steam method of target retrieval (DAKO reagent #su700). IHC experiments were performed on a DAKO autostainer following the procedures and reagents developed by DAKO. Specifically, the slides were blocked for 20 minutes (with DAKO serum free protein block #X0909), the biotin-human primary antibody applied, incubated for 2 hours at room temperature, and the slides rinsed in TBST. Vector ABC-AP (AK-5000) reagent applied. Vector Red (SK-5100) was used as a substrate. An antibody concentration of 200 Ag/ml was used. In normal tissue no staining was identified for scFv2* in the adrenal, bladder, brain, breast, colon, heart, pancreas, placenta, prostate, skin, skeletal muscle, small intestine epithelium, spleen, stomach, thymus, thyroid or uterus. Moderate staining was identified for scFv2* in the kidney, plasma cells, liver, lung, theca interna of the ovary, testis and in rare germinal center cells in the tonsils. In carcinoma, faint to moderate staining was identified for scFv2* in 2 colon cancers, 1 non-small cell lung cancer, 2 ovarian cancers, 4 prostate cancers, 7 pancreatic cancers and 4 renal cell carcinomas.

### Example 19

### FasL-Mediated Apoptosis Assay Via CALI

General: Chromophore-assisted light inactivation (CALI) can be used to inactivate proteins (e.g. PVR) on the surface of HT1080 cells. After CALI, cells were challenged with the Fas ligand (FasL) (or buffer as control) to induce apoptosis. The increase of caspase activity over 4 h compared to control buffer challenge was determined. Caspase activity was measured using a fluorogenic substrate that becomes fluorescent only after it is cleaved by active caspases. If an apoptosis-relevant protein was inactivated by CALI, no apoptosis occurs after addition of FasL. scFv1 was identified for its ability to depress the activation of caspases by FasL treatment. After CALI an enhancement of the caspase response to FasL was observed. This can be explained by the inactivation of PVR.

Experimental Set up: HT-1080 human fibrosarcoma cells were distributed into a microtiter plate (MTP) and incubated with fluorescein isothiocyanate (FITC)— conjugated scFv1. CALI uses the FITC-conjugated scFv to target incandescent light for specific protein inactivation. CALI was performed using blue-filtered diffuse light. The control experiment was carried out with dark-treated MTP. Following CALI, cells were treated with an apoptosis inducer (FasL). After a short incubation, cells were simultaneously lysed and incubated with a profluorescent substrate of caspases according to the “Homogeneous Caspase Assay” (Roche, Cat-No. 2-236-869). The amount of free rhodamine-110 cleaved reflects caspase activity, a specific early/mid-marker of apoptosis. Caspase activity was measured with an automated fluorescence MTP reader.

CALI of the Fas receptor (Fas) using the fluorescein-conjugated commercial anti-Fas antibody (UB2) caused the specific loss of function of Fas, blocking FasL-mediated signaling. The reduction in caspase activity was large for scFv1 (over 20% inhibition). There was no CALI effect without any scFv. Samples were assessed in triplicate and data were representative of three independent experiments.

1. A method of inhibiting invasiveness of a cell comprising:
contacting the cell with an antibody or fragment thereof directed against at least one extracellular domain of PVR, thereby inhibiting invasiveness of the cell.

2. The method of claim 1, wherein the cell is a cancer cell.

3. The method of claim 2, wherein the cancer cell is a fibrosarcoma, breast cancer, or glioblastoma cell.

4. A method of inhibiting adhesiveness of a cell comprising:
contacting the cell with an antibody or fragment thereof directed against at least one extracellular domain of PVR, thereby inhibiting adhesiveness of the cell.

5. The method of claim 4, wherein the cell is a cancer cell.

6. The method of claim 5, wherein the cancer cell is a fibrosarcoma, breast cancer, or glioblastoma cell.
